(19) United States
(12) Patent Application Publication
(10) Pub. No.: US 2022/0339150 A1
(43) Pub. Date: Oct. 27, 2022

(54) ENDOCANNABINOID SYSTEM-TARGETING PRODRUGS AND THERAPEUTIC USES THEREOF

(71) Applicant: MEDIPURE PHARMACEUTICALS INC., North Vancouver (CA)

(72) Inventors: Tapio NEVALAINEN, North Vancouver (CA); Rakshit D. KODEKALRA, North Vancouver (CA); Jarkko RAUTIO, North Vancouver (CA); Mahadev PATIL, North Vancouver (CA); Safwat M. RABEA, North Vancouver (CA); David Earl BOGUCKI, North Vancouver (CA); Nihar R. PANDEY, North Vancouver (CA)

(21) Appl. No.: 17/715,276

(22) Filed: Apr. 7, 2022

Related U.S. Application Data
(60) Provisional application No. 63/171,952, filed on Apr. 7, 2021.

(51) Int. Cl. A61K 31/496; A61K 31/495; A61K 31/4025; A61K 31/27; A61K 31/4453; A61K 31/4545; A61K 31/40

(52) U.S. Cl. CPC A61K 31/496; A61K 31/495; A61K 31/4025; A61K 31/27; A61K 31/4453; A61K 31/4545; A61K 31/40

(57) ABSTRACT

The present invention relates to the process and therapeutic use of endocannabinoid-targeting novel prodrugs in a delivery system. In particular, the present invention relates to prodrugs of endocannabinoid system-targeting molecules, as cannabidiol (CBD), cannabigerol (CBG), tetrahydrocannabinol (THC), and cannabichromene (CBC) for oral, transmucosal, transdermal, and parenteral administration, and methods and using the same as therapeutics.

### PRIORITY

This application claims the benefit of U.S. provisional application 63/171,952 filed Apr. 7, 2021.

### FIELD OF INVENTION

The present invention relates to the process and therapeutic use of endocannabinoid system targeting novel prodrugs in a delivery system. In particular, the present invention relates to prodrugs of the endocannabinoid system targeting molecules, as cannabidiol (CBD), cannabigerol (CBG), tetrahydrocannabinol (THC), and cannabichromene (CBC) for oral, transmucosal, transdermal, and parenteral administration, and methods and using the same as therapeutics.

### BACKGROUND OF THE INVENTION

Endocannabinoids are endogenous, substances and processes are those that originate from within a system such as an organism including tissue and cell, lipid-based retrograde neurotransmitters that bind to cannabinoid receptors (CBRs), and cannabinoid receptor proteins that are expressed throughout the vertebrate central nervous system (including the brain) and peripheral nervous system are collectively known as the endocannabinoid system (ECS).

Cannabinoids are a class of diverse chemical compounds that act on cannabinoid receptors on cells that repress neurotransmitter release in the brain. Ligands for these cannabinoid receptors include the endocannabinoids (produced naturally in the body by humans and animals), phytocannabinoids (found in cannabis and some other plants), and synthetic cannabinoids (manufactured artificially). Over 100 different cannabinoids have been isolated from cannabis, exhibiting varied effects (Pertwee, R. G. 2014. Handbook of cannabis).

The clinical usefulness of the cannabinoids to treat a range of medical conditions is well-recognized. One of the most notable phytocannabinoids, tetrahydrocannabinol (THC), is known to be effective in treating conditions such as glaucoma, AIDS, neuropathic pain, spasticity associated with multiple sclerosis, fibromyalgia, and chemotherapy-induced nausea. THC is also effective in the treatment of allergies, inflammation, infection, epilepsy, depression, migraine, bipolar disorders, anxiety disorder, drug dependency, and drug withdrawal syndromes. Cannabidiol (CBD), an isomer of THC, is a potent antioxidant and anti-inflammatory compound known to protect against acute and chronic neurodegeneration. Cannabigerol (CBG), is a cannabinoid found in high concentrations in hemp with known anti-depressant activity. Cannabichromene (CBC) possesses anti-inflammatory, anti-fungal, and anti-viral properties.

In addition to the benefits of systemically administered cannabinoids discussed above, cannabinoids have been found to have localized benefits from topical administration. For example, topically administered cannabinoids are useful to alleviate pain and other conditions originating near the surface of the skin, including but not limited to pain associated with post-herpetic neuralgia, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis and psoriatic arthritis. Topically administered cannabinoids have also been found to be useful to alleviate pain and other conditions associated with deeper tissues, such as peripheral neuropathic pain, including but not limited to the peripheral neuropathic pain associated with diabetic neuropathy, ankylosing spondylitis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, posttraumatic osteoarthritis, synovitis, and juvenile rheumatoid arthritis. It has also been found that the topical administration of cannabinoids can inhibit the growth of hair.

However, cannabinoids are highly hydrophobic compounds, and cannabinoids such as CBD, CBG, CBC, and THC exhibit a low transmucosal and transdermal permeability in mammals, such as humans. This complicates their use in drug formulations and administration of effective quantities of cannabinoids to a mammal through transmucosal and transdermal routes have been largely unsuccessful.

Moreover, cannabinoids undergo substantial first-pass metabolism when absorbed from the human gut after oral administration. Oral bioavailability is even further reduced if the patient is suffering from nausea, either due to insufficient time for a drug to get absorbed into the bloodstream to achieve a therapeutic effect, due to the dosage being expelled from the gastrointestinal tract during vomiting, or due to patients avoiding taking oral medication due to discomfort in their gut.

Thus, it would be desirable to improve their therapeutic efficacy of cannabinoids for various administration routes.

### SUMMARY OF THE INVENTION

The present invention relates to cannabinoid prodrugs. In particular, the present invention relates to prodrugs of cannabidiol (CBD), cannabigerol (CBG), tetrahydrocannabinol (THC), and cannabichromene (CBC). The cannabinoid prodrugs may contain basic partly ionized promote ties. In particular, the invention relates to carbamate and ester prodrugs of CBD, CBG, CBC, and THC.

According to an aspect of the invention, there is provided a cannabinoid prodrug having the formula:

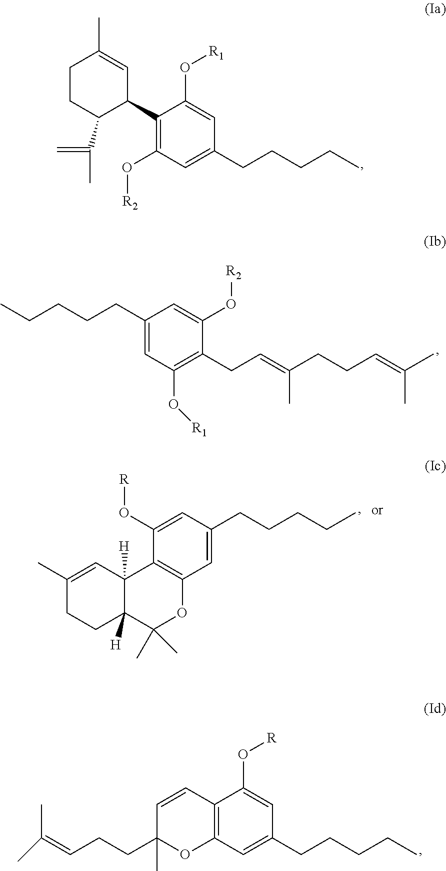

wherein:

R₁ and R₂ are each independently selected from hydrogen, alkyl, cycloalkyl, heterocycles, saturated heterocycles, and/or X, wherein X is selected from the group consisting of:

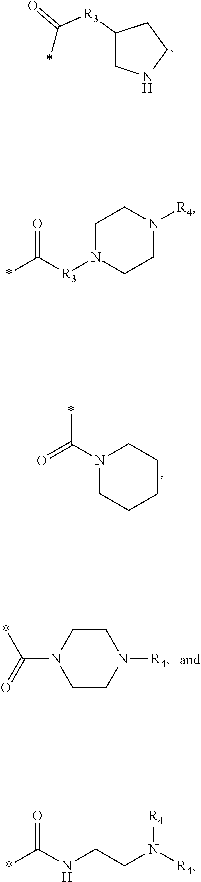

R₃ is a bond or a C₁-C₈ alkyl, alkylene or alkylidene;

R₄ is hydrogen, or a C₁-C₆ alkyl group.

In some embodiments, R₃ may be a bond, C₁-C₈ alkyl, C₁-C₈ alkylene, or C₁-C₈ alkylidene. In some embodiments, R₃ is a bond or methyl.

In some embodiments, R₄ may be hydrogen or C₁-C₆ alkyl. In some embodiments, R₄ may be hydrogen or methyl.

In some embodiments, the cannabinoid prodrug may be provided in the form of a free-base, salt, hydrate, enantiomer, isomer, tautomer, or derivative thereof.

The cannabinoid prodrug may be one of the following:

According to another aspect of the invention, there is provided a composition comprising one or more cannabinoid prodrugs described herein and one or more adjuvants, excipients, carriers, or diluents.

In some embodiments, the composition may comprise between 0.1% and about 95% by weight of the cannabinoid prodrug.

According to another aspect of the invention, there is provided a cannabinoid prodrug, or a composition comprising the cannabinoid prodrug, for use in treating a medical condition in a subject. The medical condition may be one selected from the group consisting of pain, epilepsy, an inflammatory disorder, a psychotic disorder, an optical disorder, a neurological disorder, cancer, and an immunological disorder. The cannabinoid prodrug may be administered to the subject orally, buccally, sublingually, nasally, ocularly, transmucosal, transdermally, or parenterally,

According to another aspect of the invention, there is provided a cannabinoid prodrug for use as an analgesic, an anti-convulsant, an anti-psychotic, an anti-inflammatory, an antioxidant, a neuroprotective, an anti-cancer, or an immunomodulatory agent. The cannabinoid prodrug may be administered to the subject orally, transmucosally or transdermally.

In another aspect it is provided a composition comprising a cannabinoid prodrug as described herewith, wherein the composition is for use as pesticide or insecticide.

According to another aspect of the invention, there is provided a method for treating a medical condition in a subject, the method comprising administering a cannabinoid prodrug, or a composition comprising the cannabinoid prodrug, to the subject. The medical condition may be one selected from the group consisting of pain, epilepsy, an inflammatory condition, a psychotic disorder, a neurological disorder, an optical disorder, cancer, and an immunological disorder. The cannabinoid prodrug may be administered to the subject orally, buccally, sublingually, nasally, ocularly, transmucosally, transdermally, or by parenteral administration.

According to another aspect of the invention, there is provided a method for delivery of a cannabinoid drug to a subject, comprising the step of administering a cannabinoid prodrug or a composition comprising the cannabinoid prodrug. The cannabinoid prodrug may be administered to the subject orally, transmucosally, or transdermally. The subject may be a human or an animal.

The cannabinoid prodrug or composition may be administered in a dose of from about 0.0001 mg/kg to about 2000 mg/kg. Furthermore, the cannabinoid prodrug or composition may be administered once a day or multiple times a day. In addition, the cannabinoid prodrug or composition may be administered between 0.0001-2000 mg/kg/day. The cannabinoid prodrug or composition may be for sustained, controlled or sustained and controlled release.

In another aspect it is provided a composition comprising the cannabinoid prodrug as described herewith, wherein the composition may be for use as a pesticide and/or insecticide.

According to another aspect of the invention, there is provided a method for the preparation of a cannabinoid prodrug comprising: reacting a cannabinoid with a compound comprising the moiety defined as X to obtain the cannabinoid prodrug. As described above, X may be one selected from:

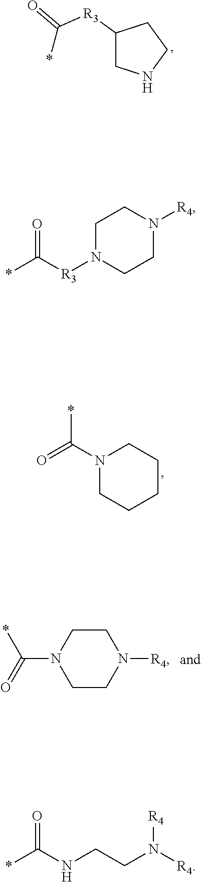

The cannabinoid may be CBD, THC, CBC, or CBG.

In yet another aspect it is provided a method of administering a compound to a subject comprising the steps of: (a) combining a cannabinoid prodrug as described herein with a pharmaceutical excipient to form a pharmaceutical composition; (b) creating a dosage form suitable for administration to a subject from the pharmaceutical composition; and (c) administering the dosage form to a subject. The subject might be a human or an animal.

In some embodiments, there is provided a method of administering a compound to a mammal comprising the steps of (a) combining one or more cannabinoid prodrug described herein with a pharmaceutical excipient to form a pharmaceutical composition; (b) creating a dosage form suitable for administration to a mammal from the pharmaceutical composition; and (c) administering the dosage form to a mammal.

Furthermore, the pharmaceutical composition may be combined with an analgesics, wherein the combination of the pharmaceutical composition and the analgesics may be a synergistic combination.

This summary of the invention does not necessarily describe all features of the invention.

### DETAILED DESCRIPTION

The following description is of a preferred embodiment of the invention.

The present description relates to cannabinoid prodrugs. In particular, the present description relates to prodrugs of cannabidiol (CBD), cannabigerol (CBG), tetrahydrocannabinol (THC), and cannabichromene (CBC). The cannabinoid prodrugs may contain basic partly ionized promoieties. In particular, the invention relates to carbamate and ester prodrugs of CBD, CBG, CBC, and THC.

Furthermore, the description provides cannabinoid prodrugs having formula (Ia), (Ib), (Ic), or (Id), as defined below. The cannabinoid prodrugs may be derived from the parent cannabinoids CBD, CBG, THC, and CBC, respectively, and may be readily hydrolyzed to the parent cannabinoid in vivo.

Advantageously, the resulting prodrugs may be more hydrophilic than their respective parent cannabinoids, and therefore more water-soluble. For example, the distribution coefficient (water/octanol partition coefficient, logD, at a pH 6.8) values for cannabinoids and their prodrugs are shown in Table 2, which demonstrates that the cannabinoid prodrugs have a lower log D value than their parent cannabinoids. The cannabinoid prodrugs of the present description were also found to be chemically stable in a non-enzyme medium and have suitable aqueous and lipid solubility meaning they are able to permeate through the biological membranes such as the buccal and transdermal membrane. For example, as shown in Examples 7 and 8, the cannabinoid prodrugs were found to have improved permeability across the buccal membrane and skin epithelium when compared with their parent cannabinoids.

Furthermore, the cannabinoid prodrugs of the present description are not significantly ionized or are not positively charged at physiological pH. Moreover, the cannabinoid prodrugs of the present description have been found to avoid the first-pass metabolism, providing greater and more prolonged levels of the cannabinoids in the bloodstream.

The improved physiochemical properties make the present cannabinoid prodrugs suitable for delivery via administration routes including oral, buccal, sublingual, injectable, topical, dermal, follicular, nasal, ocular, rectal, and vaginal.

The cannabinoid prodrugs of the present description may be defined as follows:

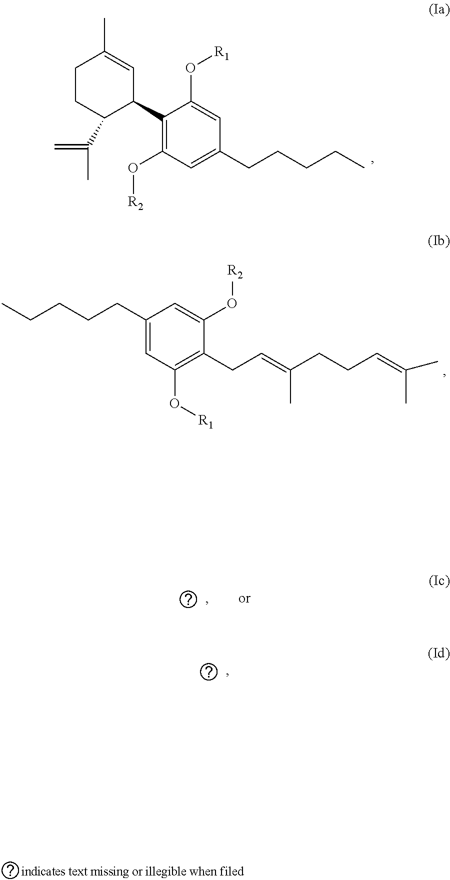

wherein:

R₁ and R₂ may each independently be selected from hydrogen and X, wherein X may be selected from the group consisting of:

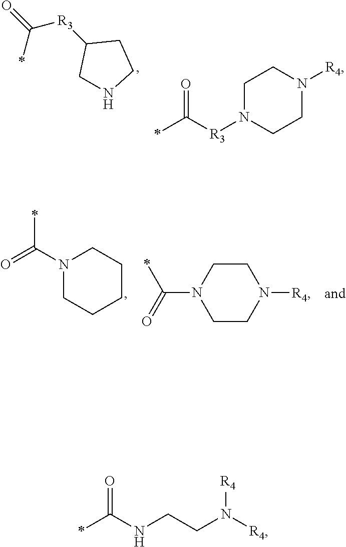

R₃ may be a bond or a C₁-C₈ alkyl, alkylene or alkylidene;

R₄ may be hydrogen, or a C₁-C₆ alkyl group.

In some embodiments, X may be

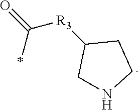

In some embodiments, X may be

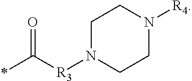

In some embodiments, X may be

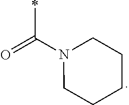 [SMILES: *C(=O)N1CCCCC1]

In some embodiments, X may be

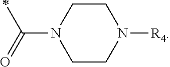

In some embodiments, X may be

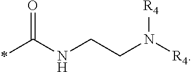

In some embodiments, R₃ may be a bond, C₁-C₈ alkyl, C₁-C₈ alkylene, or C₁-C₈ alkylidene. The C₁-C₈ alkyl, alkylene, or alkylidene may be linear, branched, or cyclic. In some embodiments, R₃ may be a bond. In some embodiments, R₃ may be C₁-C₈ alkyl. In some embodiments, R₃ may be C₁-C₈ alkylene. In some embodiments, R₃ may be C₁-C₈ alkylidene. In some embodiments, R₃ may be C₁-C₄ alkyl, C₁-C₄ alkylene, or C₁-C₄ alkylidene. In some embodiments, R₃ may be methyl, ethyl, or propyl. In some embodiments, R₃ may be methyl.

In some embodiments, R₄ may be hydrogen or C₁-C₆ alkyl. The C₁-C₆ alkyl may be linear, branched, or cyclic. In some embodiments, R₄ may be hydrogen. In some embodiments, R₄ may be C₁-C₆ alkyl. In some embodiments, R₄ may be C₁-C₃ alkyl. In some embodiments, R₄ may be methyl, ethyl, or propyl. In some embodiments, R₄ may be methyl.

In some embodiments, the cannabinoid prodrug may be provided in the form of a free-base, salt, hydrate, enantiomer, isomer, tautomer, or derivative thereof. For example, the cannabinoid prodrug may be provided as a salt of oxalic acid, citric acid, tartaric acids, lactic acid, fumaric acid, hydrochloric acid, propionic acid, sulfuric acid, nitric acid, acetic acid, maleic acid, succinic acid, or phosphoric acid. The terms “free-base”, “salt”, “hydrate”, “enantiomer”, “isomer”, “tautomer”, or “derivative” are terms known in the art.

The cannabinoid prodrug may be one of the following:

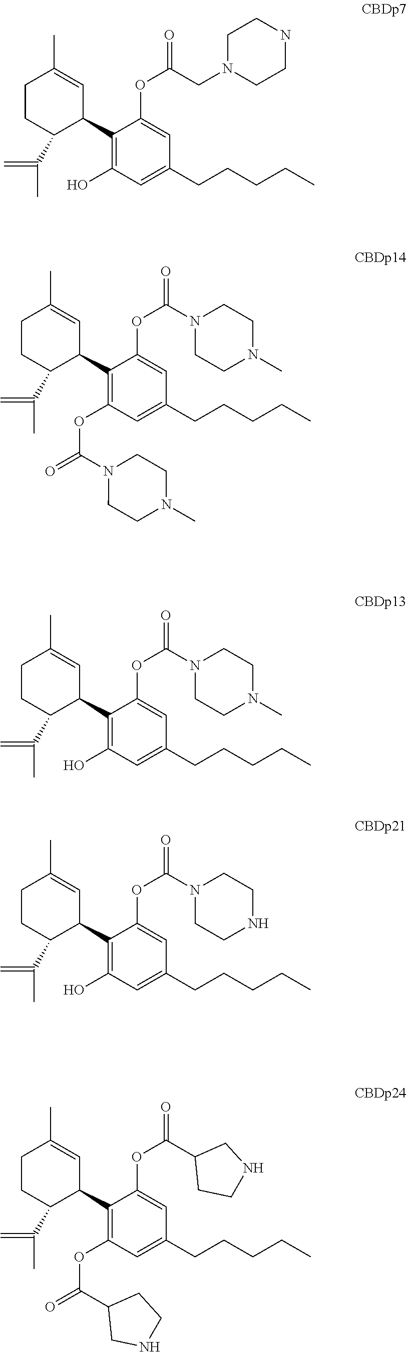

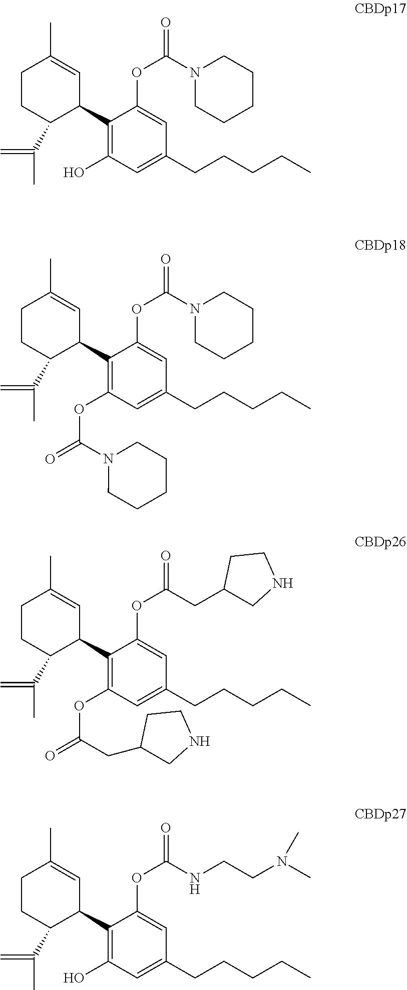

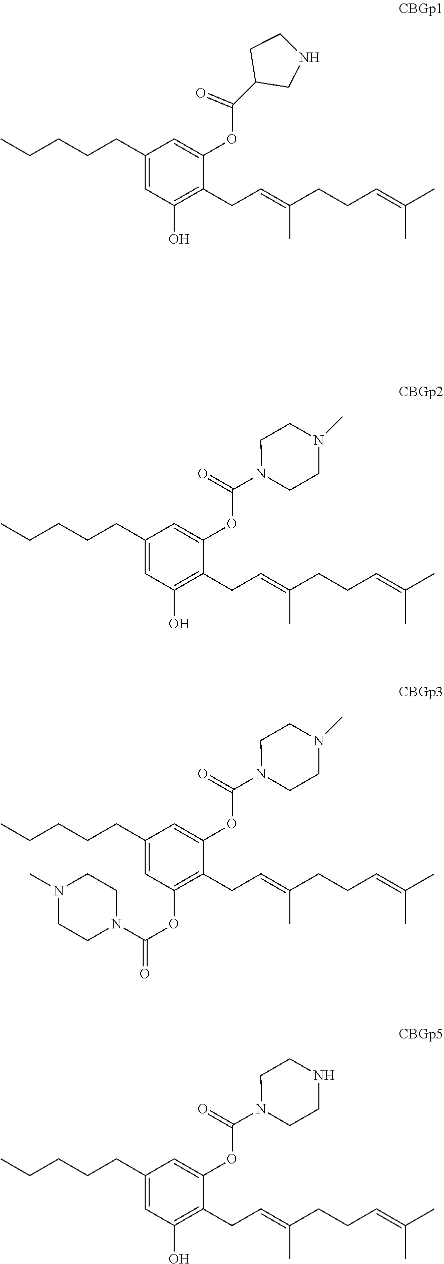

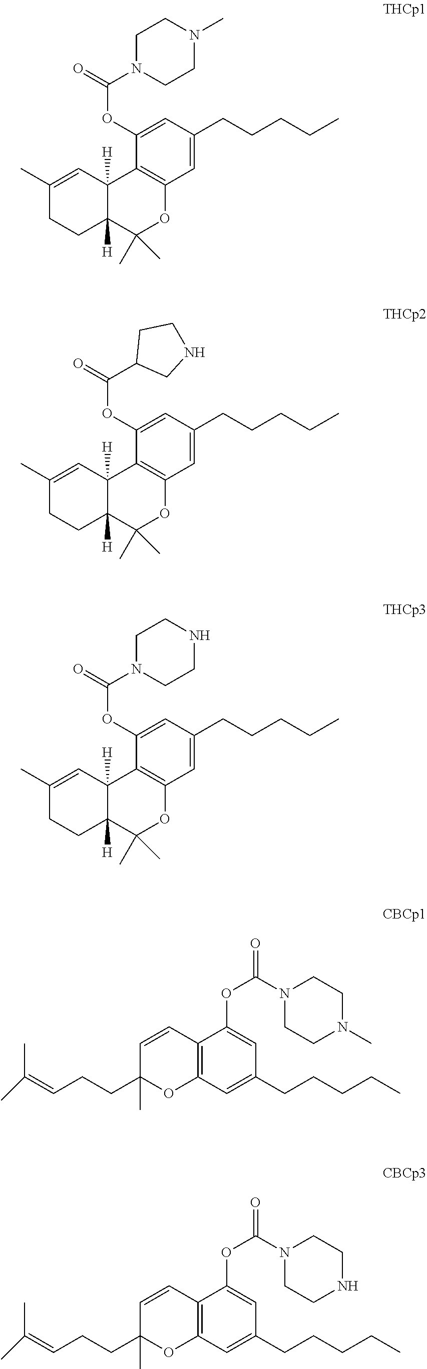

The term “alkyl” refers to straight and branched-chain alkyl groups, e.g. methyl, ethyl, propyl, isopropyl, butyl, t-butyl, pentyl, hexyl, and cyclic alkyl groups such as cyclopropane, cyclobutane, cylopentane, cyclohexane.

The term “alkylene” refers to a divalent alkane group with two hydrogen atoms removed each from different carbon atoms, e. g. a C₂ alkylene would have the structure *—C═C—*.

The term “alkylidene” refers to a divalent alkane group with two hydrogen atoms removed from the same carbon, e.g. a C₂ alkylidene would have the structure *═C—C—*.

### Parent Cannabinoids

The term “parent cannabinoid” refers to the cannabinoid that the prodrug is converted to once metabolized. The cannabinoid prodrugs of the present description may be derivatives of a parent cannabinoid or analog.

Accordingly, the prodrug described herewith may be a derivative of the following non-limiting parent cannabinoids:

### Cannabidiol (CBD)

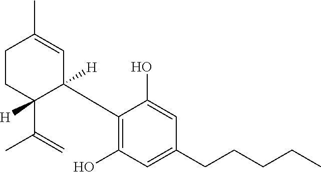 [SMILES: C=C(C)[C@@H]1CCC(C)=C[C@H]1c1c(O)cc(CCCCC)cc1O]

Cannabidiol (CBD) is a phytocannabinoid derived from Cannabis, which is devoid of psychoactive activity, with analgesic, anti-inflammatory, antineoplastic and chemopreventive activities. Upon administration, cannabidiol (CBD) exerts its anti-proliferative, anxiolytic, anti-angiogenic and pro-apoptotic activity. Moreover, several such studies have shown that CBD is effective for the treatment of epilepsy. Epidiolex is a CBD drug, which has been approved for the treatment of seizures associated with Lennox-Gastaut syndrome or Dravet syndrome in patients 2 years of age and older (https://www.accessdata.fda.gov/drugsatfda_docs/label/2018/210365lbl.pdf).

### Cannabigerol (CBG)

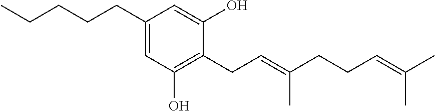 [SMILES: CCCCCc1cc(O)c(C/C=C(\C)CCC=C(C)C)c(O)c1]

Cannabigerol (CBG) is a cannabinoid found in Cannabis plants. It has been found to act as a α2-adrenergic receptor agonist. It is a moderately potent 5HT1A receptor antagonist and low-affinity CB1/CB2 receptor antagonist. CBG may have potential use for alleviating pain and anxiety, inflammation, digestive conditions, skin conditions, glaucoma, and autoimmune diseases. In addition, it has been shown to have antimicrobial activity and neuroprotective properties, which may be promising for the treatment of neurodegenerative diseases such as Huntington's disease and multiple sclerosis. CBG may also have potential in the treatment of cancers.

### Cannabichromene (CBC)

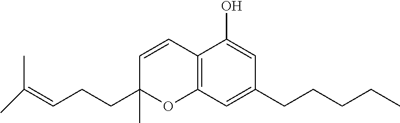 [SMILES: CCCCCc1cc(O)c2c(c1)OC(C)(CCC=C(C)C)C=C2]

Cannabichromene (CBC) is a cannabinoid found in Cannabis plants. CBC is known to interact with the TRPV1 and TRPA1 receptors. It may play a role in the anti-inflammatory and anti-viral effects of cannabis, and it may have also antifungal properties. Cannabichromene may contribute to the overall analgesic effects of medical cannabis. Moreover, CBC may have antidepressant effects and it may promote neurogenesis.

### Δ9-Tetrahydrocannabinol (THC)

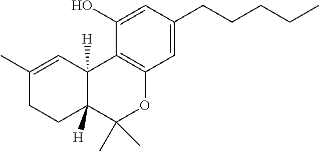 [SMILES: CCCCCc1cc(O)c2c(c1)OC(C)(C)[C@@H]1CCC(C)=C[C@@H]21]

Δ9-Tetrahydrocannabinol (THC) is the primary active ingredient of the Cannabis sativa plant and is responsible for most of the psychoactive effects at higher doses, and pharmacological effects at lower non-psychoactive doses. The most promising clinical applications are for the control of nausea and vomiting associated with chemotherapy and for appetite stimulation of AIDS patients suffering from anorexia and wasting syndrome. However, THC has demonstrated other biological activities which can be used in therapeutic applications like analgesic, glaucoma, migraine, spasticity/epileptic seizures, anxiety and chemical dependence withdrawal symptoms. Nabiximols and Sativex, are oromucosal sprays, containing delta-9-tetrahydrocannabinol (THC) and cannabidiol (CBD) in a 1:1 ratio for the treatment of spasticity due to multiple sclerosis, pain, and chronic cancer pain.

### Cannabinoid Prodrugs

The term “prodrug” refers to a pharmacologically inactive derivative of an active drug or drug molecule. The prodrug may be converted to an active drug after administration. For example, a prodrug may refer to a compound that undergoes a chemical conversion (for example hydrolysis or cleavage of moiety), through a metabolic process or otherwise, to release the active (free) drug.

Only a limited number of cannabinoid prodrugs have been reported in the literature. For example, WO 2009/018389 describes cannabidiol prodrugs with improved solubility, WO 2011/026144 describes cannabidiol prodrugs in topical and transdermal administration with microneedles, WO 2017053574 describes cannabinoid glycoside prodrugs, and WO2017132526 describes cannabidiol esters. Furthermore, THC esters (WO 2000/029402) and amino acid esters (WO 2010/051541) of THC including THC-valine-hemisuccinate are known, which have shown enhanced ocular penetration (Invest Ophthalmol Vis Sci. 2017 April; 58 (4): 2167-2179).

Without wishing to be bound by theory, it is believed that prodrugs that are partially ionized at the oral mucosal pH of 6.8, are more soluble in the oral cavity after insertion of the buccal formulation. A good transmucosal prodrug should have sufficient solubility in the buccal cavity, which is combined with sufficient lipophilicity for passage through mucosal membranes.

### Synthesis of Cannabinoid Prodrugs

The cannabinoid prodrugs may be synthesized by reacting a cannabinoid or parent cannabinoid with a compound comprising the X moiety to obtain the cannabinoid prodrug. The cannabinoid may be for example CBD, THC, CBC, or CBG.

For example, ester cannabinoid prodrugs may be prepared by (a) reacting a cannabinoid with a protected acid moiety to form a protected ester cannabinoid; and (b) deprotecting to produce the cannabinoid prodrug. A suitable protecting agent may be N-Boc. DMAP may be used as a catalyst. The reaction may be performed in dry dichloromethane. Deprotection may be performed in acidic conditions.

Carbamate cannabinoid prodrugs may be prepared by (a) treating with phosgene or triphosgene e.g. under basic conditions to generate a cannabinoid chloroformate; (b) coupling to a protected moiety such as piperazine; (c) deprotecting to produce the cannabinoid prodrug. A suitable protecting agent may be N-Boc. Deprotection may be performed in acidic conditions. Alternatively, the cannabinoid may be reacted directly with a chloroformate moiety to produce the cannabinoid prodrug.

Alternatively, carbamate cannabinoid prodrugs may be prepared by (a) treating an amine with 1,1′-carbonyldiimidazole to generate a presumed 1H-imidazole-1-carboxamide; (b) reacting with a cannabinoid to produce the cannabinoid prodrug.

The cannabinoid prodrug may then be washed and extracted from an organic phase.

The starting material (starting cannabinoid) may be derived from a plant, microbial synthesis, or may be derived synthetically. Alternatively, the cannabinoid prodrugs may be derived synthetically using known synthetic chemistry methods.

### Pharmaceutical Compositions

The compositions described herein may be pharmaceutical compositions and may include one or more pharmaceutically acceptable excipient or adjuvant, e.g. to achieve a composition usable as a dosage form.

The term “excipient” refers to any substance, not itself a therapeutic agent, used as a carrier or vehicle for delivery of a therapeutic agent to a subject or combined with a therapeutic agent (e.g., to create a pharmaceutical composition) to improve its handling or storage properties or to permit or facilitate the formation of a dose unit of the composition. Pharmaceutically acceptable excipients include, by way of illustration and not limitation, binders, disintegrants, taste enhancers, diluents, solvents, thickening agents, penetration enhancers, wetting agents, lubricants, emollients, substances added to mask or counteract a disagreeable odor, fragrances, or taste, and substances added to improve appearance or texture of the composition. The term “adjuvant” refers to a pharmacological agent that improves the efficacy of the therapeutic agent.

The cannabinoid prodrugs may be formulated in a composition suitable for oral, buccal, sublingual, injectable, topical, follicular, nasal, ocular, rectal, or vaginal delivery. In some embodiments, the compositions comprising cannabinoid prodrugs are formulated for oral, transmucosal (buccal/sublingual), or transdermal use. In some embodiments, the cannabinoid prodrug is administered orally. In some embodiments, the cannabinoid prodrug is administered transmucosally. In some embodiments, the cannabinoid prodrug is administered transdermally. The compositions according to the description may be in different forms, in particular a form chosen from the group comprising tablets, capsules, pills, syrups, suspensions, solutions, powders, granules, emulsions, microspheres and injectable solutions, and solid lipid nanoparticles.

In some embodiments, the compositions described herein are suitable for transdermal administration. Transdermally administrable compositions may be adapted for administration in and/or around the abdomen, back, chest, legs, arms, scalp, or other suitable skin surface and may include formulations in which the cannabinoid prodrug is administered in patches, ointments, creams, suspensions, lotions, pastes, gels, sprays, foams or oils. Transdermally administrable compositions include formulations in which the cannabinoid prodrug is placed in a glycol or gel formulation.

In some embodiments, the compositions described herein are suitable for topical administration. Topically administrable compositions are adapted for administration in and/or around the abdomen, back, chest, legs, arms, scalp, or other suitable skin surface and may include formulations in which the cannabinoid prodrug is administered in patches, ointments, creams, suspensions, lotions, pastes, gels, sprays, foams or oils.

In some embodiments, the compositions described herein are suitable for oral administration. Orally administrable compositions include formulations in which the cannabinoid prodrug is administered in tablets, capsules, suspensions, syrups, or liquids. In an additional embodiment, the composition may be formulated as an extended-release or long-acting tablet or capsule. The oral dosage form may be enteric-coated using techniques known to a person of ordinary skill in the art.

In some embodiments, the compositions described herein are suitable for buccal administration. Bucally administrable compositions may include formulations in which the cannabinoid prodrug is administered in lozenges, sprays, gels, pastes, dissolvable tablets, or dissolvable strips.

In some embodiments, the compositions described herein are suitable for sublingual administration. Sublingually administrable compositions may include formulations in which the cannabinoid prodrug is administered in lozenges, sprays, gels, pastes, dissolvable tablets, or dissolvable strips.

In some embodiments, the compositions described herein are suitable for injectable administration. Injectable administrable compositions may include formulations in which the cannabinoid prodrug is administered as an intravenous, intrathecal, subcutaneous, or depot injection.

In some embodiments, the compositions described herein are suitable for rectal administration. Rectally administrable compositions may include formulations in which the cannabinoid prodrug is placed in suppositories, ointments, creams, suspensions, solutions, lotions, pastes, gels, sprays, foams, or oils.

In some embodiments, the compositions described herein are suitable for vaginal administration. Vaginally administrable may include formulations in which the cannabinoid prodrug is placed in suppositories, ointments, creams, suspensions, solutions, lotions, pastes, gels, sprays, foams, or oils.

In some embodiments, the compositions described herein are suitable for ocular administration, Ocularly administrable compositions may include formulations in which the cannabinoid prodrug is placed in ointments, suspensions, solutions, gels, or sprays.

In some embodiment, the compositions described herein are suitable for nasal administration. Nasally administrable may include formulations in which the cannabinoid prodrug is placed in ointments, suspensions, solutions, lotions, pastes, gels, sprays, or mists.

The cannabinoid prodrug or composition as described herewith may be administered to a subject as described above. The subject may be a human or an animal. Accordingly, the cannabinoid drug or composition as described herewith may be used in human and/or veterinary medicine. For example, the animal may be a mammal, bird, fish, or reptile. Non-limiting examples of animals include dogs, cats, rats, guinea pigs, rabbits, and the like, and farm animals, such as horses, cows, pigs, hogs, cattle, goats, sheep, poultry and the like.

In another aspect the cannabinoid prodrug or composition as described herewith may be combined with an analgesics, wherein the combination of the pharmaceutical composition and the analgesics is a synergistic combination. By “synergistic,” it is meant that the combination of two or more agents, for example, the combination of the cannabinoid prodrug or composition and the analgesics yield a combination index (CI)<1.0. CI for the drug combinations when determined with the Chou-Talalay combination index method using CompuSyn software (Chou, T. C., Martin, N., CompuSyn for drug combinations: PC software and user's guide, A Computer Program for Quantitation of Synergism and Antagonism in Drug Combinations, and the Determination of IC50 and ED50 and LD50 Values. CompuSyn, PD Science. 2005, Paramus, N.J. 7652-1754). The drug combination may be considered synergism if CI<1, antagonism if CI>1, and additive effect if CI=1.

### Dosages

The compositions of the present description may comprise a therapeutically or prophylactically effective amount of one or more cannabinoid prodrugs.

The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

A therapeutically effective amount may vary according to factors such as the disease state, age, sex and weight of the subject, and the ability of the perborate salt to elicit a desired response in the subject. Dosage regimens may be adjusted to provide the optimum therapeutic response. A therapeutically effective amount is also one in which any toxic or detrimental effects of the formulation are outweighed by the therapeutically beneficial effects. A “prophylactically effective amount” refers to an amount effective, at dosages and for periods necessary, to achieve the desired prophylactic result, such as the prevention or the prevention of the progression of cancer. Typically, a prophylactic dose is used in subjects before or at an earlier state of disease.

It is to be noted that dosage values may vary with the severity of the condition to be alleviated. For any particular subject, specific dosage regimens may be adjusted over time according to the individual need and the professional judgment of the person administering or supervising the administration of the compositions. For example, a single bolus may be administered, several divided doses may be administered over time or the dosage may be proportionally reduced or increased as indicated by the exigencies of the therapeutic situation. It may be advantageous to formulate compositions in dosage unit form for ease of administration and uniformity of dosage.

For example, the compositions of the description may comprise between about 0.1% and about 95% by weight of one or more cannabinoid prodrugs. The compositions may comprise between about 0.1% to about 50% (wt/wt) of one or more cannabinoid prodrugs. The compositions may comprise between about 0.1% to about 40% (wt/wt) of one or more cannabinoid prodrugs. The compositions may comprise between about 5% to about 30% (wt/wt) of one or more cannabinoid prodrugs. The compositions may comprise between about 5% to about 20% (wt/wt) of one or more cannabinoid prodrugs. The compositions may comprise between about 10% to about 20% (wt/wt) of one or more cannabinoid prodrugs.

The dose of the one or more cannabinoid prodrugs may comprise from about 0.0001 mg/kg to about 2000 mg/kg or any amount therebetween. For example, the dose of c may be 0.0001, 0.001, 0.01, 0.1, 1, 5, 10, 15, 25, 50 100, 150, 250, 500, 750, 1000, 1500, 2000 mg/kg or any amount therebetween.

Furthermore, the composition comprising the one or more than one cannabinoid prodrug may be administered in a dose of from between 0.0001-2000 mg/kg or any amount therebetween. For example, the composition may be administered at 0.0001, 0.001, 0.01, 0.1, 1, 5, 10, 15, 25, 50 100, 150, 250, 500, 750, 1000, 1500, 2000 mg/kg or any amount therebetween. Furthermore, the composition may be administered in a dose of from between 0.0001-2000 mg/kg/day or any amount therebetween. For example, the composition may be administered at 0.0001, 0.001, 0.01, 0.1, 1, 5, 10, 15, 25, 50 100, 150, 250, 500, 750, 1000, 1500, 2000 mg/kg/day or any amount therebetween.

The cannabinoid prodrug or the composition comprising the cannabinoid prodrug may be administered in a dose once a day or multiple times a day. For example, the cannabinoid prodrug or the composition comprising the cannabinoid prodrug may be administered once, twice, three, four, five, or six times a day.

The daily dose may be between 0.0001-20 mg/day or any amount therebetween. In another example, the maximum daily dose may be between 1 mg/day-10 g/day or any amount therebetween.

Furthermore, the cannabinoid prodrug or the composition comprising one or more than one cannabinoid prodrug may be combined with other molecules, intermediates, prodrugs, drugs or precursors to be administered to a subject in need thereof.

The compositions of the present description may be used alone or in combination with other biologically active ingredients. A composition of the present invention, alone or in combination with other active ingredients, may be administered to a subject in a single dose or multiple doses over a period of time.

### Therapeutic Uses

The cannabinoid prodrugs may be used to treat a variety of medical conditions including but not limited to pain, epilepsy, inflammatory disorders, psychotic disorders, neurological disorders, ocular disorders, cancer, and immunological disorders.

Pain includes but is not limited to, neuropathic pain, inflammatory pain, or nociceptive pain. The pain may be acute pain, chronic pain, breakthrough pain, bone pain, soft tissue pain, nerve pain, referred pain, phantom pain, and/or cancer pain.

Inflammatory disorders include but are not limited to, rheumatoid arthritis (RA), autoimmune conditions, inflammatory bowel diseases (including Crohn's disease and ulcerative colitis), non-healing wounds, multiple sclerosis, cancer, atherosclerosis, sjogrens disease, diabetes, lupus erythematosus (including systemic lupus erythematosus), asthma, fibrotic diseases (including liver cirrhosis), pulmonary fibrosis, UV damage, psoriasis, psoriatic arthritis, ankylosing spondylitis, myositis and/or cardiovascular disease.

Psychotic disorders include but are not limited to, schizophrenia, affective psychosis, delusional disorder, bipolar disorder, depression, and/or anxiety disorder.

Neurological disorders include but are not limited to, depression, mania, and other mood disorders, bipolar disorder, obsessive-compulsive disorder (OCD), Tourette's syndrome, schizophrenia, dissociative disorders, anxiety disorders, phobic disorders, post-traumatic stress disorder (PTSD), borderline personality disorder, Attention-Deficit/Hyperactivity Disorder (ADHD), addiction disorders, seizure disorders such as epilepsy, neurodegenerative disorders such as Alzheimer's Disease, Parkinson's Disease, Huntington's disease, Amyotrophic Lateral Sclerosis, peripheral neuropathy, and/or Creutzfeldt-Jacob disease.

Ocular disorders include but are not limited to, glaucoma, macular degeneration, diabetic retinopathy, choroidal neovascularization, proliferative vitreoretinopathy, blepharitis, meibomitis, and/or dry eye syndrome.

Cancer includes but is not limited to, prostate, pancreatic, breast, lung, gastric, ovarian, uterine, colorectal, liver, bladder, stomach, pulmonary, melanoma, lymphoma, mesothelioma, medulloblastoma, glioma, and/or AML.

Immunological disorders include but are not limited to, allergic diseases such as asthma, allergic rhinitis, eczema, atopic dermatitis or allergic contact dermatitis, autoimmune diseases such as rheumatoid arthritis, systemic lupus erythematosus, Graves' disease, immune thrombocytopenic purpura, myasthenia gravis, ulcerative colitis, Crohn's disease, scleroderma and/or psoriasis.

The cannabinoid prodrugs may be used for the analgesic, anti-convulsant, anti-psychotic, anti-inflammatory, antioxidant, neuroprotective, anti-cancer, and/or immunomodulatory properties of the parent cannabinoid.

For example, the cannabinoid prodrugs may be used to treat glaucoma, AIDS wasting, neuropathic pain, post-surgical pain management, diabetic neuropathy, metabolic syndrome, fatty liver diseases & stenosis including NASH, multiple sclerosis, fibromyalgia, chemotherapy-induced nausea, allergies, inflammation, infection, Psoriasis, Eczema, and other inflammatory skin conditions, epilepsy, stress, depression, migraine, panic attacks, PTSD, bipolar disorders, anxiety disorder, drug dependency, and drug withdrawal syndromes, to provide protection against acute and chronic neuro-degeneration, or to provide anti-inflammatory, anti-fungal and anti-viral properties. The cannabinoid prodrugs may be formulated for oral, sublingual, topical, transdermal, transmucosal, or parenteral administration to deliver a therapeutically effective amount of the cannabinoid to the patient in need of treatment. For example, the cannabinoid prodrug may be formulated as pills, dragee, tablets, capsules, thin-film, gel, liquids, syrup, hydrogel, edibles, inhalers, injectables (IV, IP, IM, SC, etc.), patch, or epidural.

The cannabinoid prodrugs described herein may also be used to treat various skin conditions, including but not limited to pain associated with post-herpetic neuralgia, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis and/or psoriatic arthritis. For skin conditions, the cannabinoid prodrugs may be formulated for topical (transdermal) administration. Topically administered cannabinoid prodrugs may also be used to alleviate pain and other conditions associated with deeper tissues, such as peripheral neuropathic pain, including but not limited to the peripheral neuropathic pain associated with diabetic neuropathy, ankylosing spondylitis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, posttraumatic osteoarthritis, synovitis, and/or juvenile rheumatoid arthritis.

If the cannabinoid prodrugs are to be administered topically to treat pain and other conditions associated with deeper tissues, such as peripheral neuropathic pain, the cannabinoid prodrugs may also be co-administered systemically as well as topically to improve the therapeutic result.

The description provides methods for delivering a cannabinoid to a subject by administering the cannabinoid prodrugs described herein. The description also provides methods of administering a cannabinoid compound to a subject comprising the steps of: (a) combining a cannabinoid prodrug with a pharmaceutical excipient to form a pharmaceutical composition; (b) creating a dosage form suitable for administration to a subject from the pharmaceutical composition; and (c) administering the dosage form to a subject. For example, the subject may be a mammal such as a human. Furthermore, the subject may also be an animal. For example, the animal may be a domestic animal or a zoo animal. Non-limiting examples of animals include cattle, horse, sheep, goat, fish, birds such as chicken, turkey, duck, or goose, as well as pets, such as dogs, cats, or rodents.

In another aspect a composition comprising the cannabinoid prodrug as described may be for use as a pesticide and/or insecticide. Without wishing to be bound by theory, it is know that cannabinoids may repel insects and inhibit the growth of microbial pathogens (see for example Park, S H. et al. Sci Rep 9, 10481 (2019, which is incorporated herewith by reference). Therefore it is provided a pesticide and/or insecticide comprising the cannabinoid prodrug as described herewith.

As used herein, the terms “comprising,” “having,” “including” and “containing,” and grammatical variations thereof, are inclusive or open-ended and do not exclude additional, un-recited elements and/or method steps. The term “consisting of” when used herein in connection with use or method, excludes the presence of additional elements and/or method steps. A use or method described herein as comprising certain elements and/or steps may also, in certain embodiments consist essentially of those elements and/or steps, and in other embodiments consist of those elements and/or steps, whether or not these embodiments are specifically referred to. In addition, the use of the singular includes the plural, and “or” means “and/or” unless otherwise stated. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

The present description will be further illustrated in the following examples.

### EXAMPLES

### Example 1: Synthesis of Cannabinoid Prodrugs

Cannabinoid ester prodrugs were prepared in two steps. In the first step, the cannabinoid was reacted with an N-Boc protected acid using coupling reagent such as N,N′-dicyclohexylcarbodiimide (DCC) or N,N′-diisopropylcarbodiimide (DIC), and 4-(N,N-dimethylamino) pyridine (DMAP) as a catalyst in dry dichloromethane to obtain an N-Boc protected ester and then subsequently deprotected in acidic conditions (e.g., 50% trifluoroacetic acid in dichloromethane or hydrochloric acid in a suitable solvent such as cyclopentyl methyl ether) to obtain the cannabinoid prodrugs. After completion of the reaction, the reaction mixture was washed with saturated aqueous NaHCO₃ and extracted with dichloromethane (CH₂Cl₂). The organic layer was separated, evaporated, and dried on a rotary evaporator to give the mono and/or bis prodrug of cannabinoid.

Cannabinoid carbamate prodrugs were synthesized by treatment with phosgene (COCl₂) or triphosgene under basic conditions (e.g., triethylamine) to generate the chloroformate, followed by coupling with N-methylpiperazine or N-Boc-piperazine in the presence of a base (such as trimethylamine) and then cleavage of Boc protecting groups in acidic conditions (e.g. 50% trifluoroacetic acid in dichloromethane or 3 M HCl in cyclopentyl methyl ether). Alternatively, carbamate cannabinoid prodrugs were synthesized by treating an amine with 1,1′-carbonyldiimidazole to generate a presumed 1H-imidazole-1-carboxamide followed by reacting with a cannabinoid.

Synthesis products were purified by automated flash chromatography (BÜCHI Sepacore™ Flash) using ethyl acetate (EtOAc)-petroleum ether or dichloromethane (CH₂Cl₂)-methanol (MeOH) solvent mixture. Dichloromethane (CH₂Cl₂) was dried over activated 3 Å molecular sieves; all other solvents and reagents were used from commercial sources without further purifications.

The identity and purity of each prodrug was determined by HPLC, mass spectra, and NMR. Silica gel (200-300 mesh) was used for column chromatography. Thin-layer chromatography (TLC) was carried out on GF254 plates (0.25 mm layer thickness). ¹H NMR and ¹³C NMR experiments were performed on Bruker AVANCE 500 DRX or Bruker Avance III HD 600 spectrometer at ambient temperature. The residual solvent protons (¹H) or the solvent carbons (¹³C) were used as internal standards. ¹H NMR data are presented as follows: a chemical shift in ppm downfield from tetramethylsilane (multiplicity, coupling constant, integration). Chemical shifts (δ) are provided in ppm with reference to solvent signals ¹H NMR: CDCl₃ (7.26), MeOH-d₄ (3.31), DMSO-d₆ (2.50); ¹³C NMR: CDCl₃ (77.0), MeOH-d₄ (49.15), DMSO-d₆ (39.52). The following abbreviations are used in reporting NMR data: s, singlet; br s, broad singlet; br, broad signal; d, doublet; t, triplet; m, multiplet; q, quintet.

### Synthesis of CBDp7: (1′R,2′R)-6-hydroxy-5′-methyl-4-pentyl-2′-(prop-1-en-2-yl)-1′,2′,3′,4′-tetrahydro-[1,1′-biphenyl]-2-yl 2-(4-methylpiperazin-1-yl)acetate

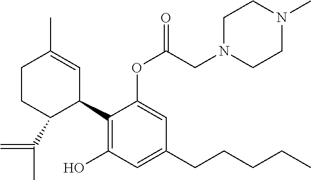 [SMILES: C=C(C)[C@@H]1CCC(C)=C[C@H]1c1c(O)cc(CCCCC)cc1OC(=O)CN1CCN(C)CC1]

(−)-Cannabidiol (500 mg, 1.59 mmol) and 2-(4-methylpiperazine-1-yl) acetic acid (300 mg, 0.189 mmol) were dissolved in dry dichloromethane (5 mL), and the solution was heated to reflux until solid precipitated (approximately 15 minutes). To this mixture was added 4-(dimethylamino)pyridine (233 mg, 1.91 mmol), and the mixture was stirred for 10 minutes at ambient temperature. N,N′-Dicyclohexylcarbodiimide (394 mg, 1.91 mmol) was added, and the mixture was stirred for 24 hours at ambient temperature then concentrated in vacuo. The residue was purified by column chromatography, eluting with a gradient of 20% to 40% ethyl acetate in petroleum ether to afford the title compound as a colourless viscous oil (303 mg, 42% yield).

¹H NMR (600 MHz, CDCl₃): δ 6.54 (br s, 1H), 6.34 (br s, 1H), 6.30-6.00 (br s, 1H), 5.51 (br s, 1H), 4.58 (br s, 1H), 4.44 (br s, 1H), 3.47 (m, 1H), 3.44-3.32 (m, 2H), 2.82-2.42 (m, 8H), 2.45-2.40 (m, 1H), 2.43 (t, J=7.7 Hz, 2H), 2.32 (s, 3H), 2.21 (m, 1H), 2.06 (m, 1H), 1.83-1.79 (m, 2H), 1.75 (s, 3H), 1.61 (s, 3H), 1.56 (m, 2H), 1.34-1.28 (m, 4H), 0.87 (t, J=7.0 Hz, 3H); ¹³C NMR (150 MHz, CDCl₃): δ 168.5, 155.6, 148.8, 147.0, 142.8, 140.1, 123.1, 118.8, 114.6, 113.8, 111.3, 59.0, 54.7 (2C), 52.8 (2C), 45.8, 45.5, 37.8, 35.3, 31.4, 30.5, 30.1, 27.8, 23.6, 22.4, 19.8, 14.0.

### Synthesis of CBDp14: (1′R,2′R)-5′-methyl-4-pentyl-2′-(prop-1-en-2-yl)-1′,2′,3′,4′-tetrahydro-[1,1′-biphenyl]-2,6-diyl bis(4-methylpiperazine-1-carboxylate)

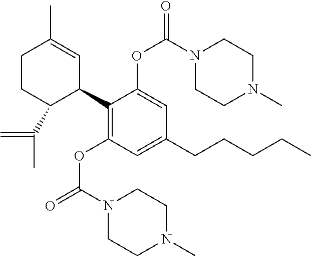 [SMILES: C=C(C)[C@@H]1CCC(C)=C[C@H]1c1c(OC(=O)N2CCN(C)CC2)cc(CCCCC)cc1OC(=O)N1CCN(C)CC1]

To a stirred solution of (−)-cannabidiol (250 mg, 0.795 mmol) and 4-methyl-1-piperazinecarbonyl chloride (387 mg, 2.37 mmol) in dichloromethane (5 mL) was added 4-(dimethylamino)pyridine (100 mg, 0.818 mmol) followed by triethylamine (240 mg, 2.37 mmol). The mixture was stirred at ambient temperature overnight then diluted with dichloromethane (25 mL) and washed with water. The organic layer was concentrated in vacuo, and the residue was purified by column chromatography, eluting with 10% methanol in dichloromethane to afford the title compound as a light amber, viscous oil (275 mg, 61% yield).

¹H NMR (600 MHz, CDCl₃): δ 6.72 (s, 2H), 5.33 (s, 1H), 4.56 (br s, 1H), 4.48 (br s, 1H), 3.74-3.40 (m, 9H), 2.52 (t, J=7.9 Hz, 2H), 2.50-2.36 (m, 9H), 2.34 (s, 3H), 2.14-1.98 (m, 2H), 1.81-1.68 (m, 2H), 1.75 (s, 3H), 1.64 (s, 3H), 1.59 (m, 2H), 1.53 (s, 3H), 1.33-1.27 (m, 4H), 0.87 (t, J=6.9 Hz, 3H); ¹³C NMR (150 MHz, CDCl₃): δ 153.2 (2C), 150.0 (2C), 147.8, 141.7, 131.9, 126.6, 125.0, 120.7, 119.6, 110.9, 54.9 (2C), 54.6 (2C), 46.2 (2C), 46.1, 44.8 (2C), 44.3 (2C), 38.5, 35.3, 31.6, 30.7, 30.5, 28.9, 23.3, 22.4, 20.1, 14.0.

### Synthesis of CBDp21: (1′R,2′R)-6-hydroxy-5′-methyl-4-pentyl-2′-(prop-1-en-2-yl)-1′,2′,3′,4′-tetrahydro-[1,1′-biphenyl]-2-yl piperazine-1-carboxylate

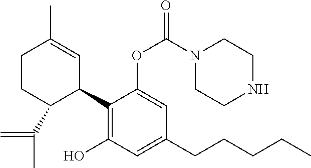 [SMILES: C=C(C)[C@@H]1CCC(C)=C[C@H]1c1c(O)cc(CCCCC)cc1OC(=O)N1CCNCC1]

Triphosgene (150 mg, 0.505 mmol) was added to a solution of (−)-cannabidiol (500 mg, 1.59 mmol) and triethylamine (161 mg, 1.59 mmol) in dichloromethane at 0° C. The mixture was stirred at 0° C. for 15 minutes then at ambient temperature for 3 hours. Saturated aqueous sodium bicarbonate was added, and the aqueous phase was extracted with dichloromethane (50 mL). The combined organic layers were dried over anhydrous sodium sulfate, filtered and concentrated in vacuo to provide (1′R,2′R)-6-hydroxy-5′-methyl-4-pentyl-2′-(prop-1-en-2-yl)-1′,2′,3′,4′-tetrahydro-[1,1′-biphenyl]-2-yl carbonochloridate as an oil (350 mg) that was used in the next step without further purification.

Piperazine (75 mg, 0.87 mmol) was added to a stirred solution of the oil from above (350 mg, 0.928 mmol) in dichloromethane, and the mixture was stirred at ambient temperature overnight. The mixture was concentrated in vacuo, and the residue was purified by column chromatography, eluting with 10% methanol in dichloromethane to afford the title compound as a light pink solid (360 mg, 53% yield).

¹H NMR (600 MHz, CDCl₃): δ 6.52 (br s, 1H), 6.42 (br s, 1H), 5.59 (br s, 1H), 4.61 (br s, 1H), 4.47 (br s, 1H), 3.65-3.40 (m, 5H), 2.90 (m, 4H), 2.60-2.36 (m, 4H), 2.21 (m, 1H), 2.06 (m, 1H), 1.83-1.68 (m, 5H), 1.63-1.52 (m, 5H), 1.34-1.28 (m, 4H), 0.87 (t, J=7.0 Hz, 3H) (OH not observed); ¹³C NMR (150 MHz, CDCl₃): δ 155.6, 153.6, 149.7, 147.3, 142.7, 140.1, 123.5, 119.6, 114.3 (2C), 111.0, 45.9, 45.7, 45.3, 44.8 (2C), 37.9, 35.4, 31.4, 30.5, 30.0, 27.6, 23.6, 22.6, 20.3, 14.0.

### Synthesis of CBDp24: (1′R,2′R)-5′-methyl-4-pentyl-2′-(prop-1-en-2-yl)-1′,2′,3′,4′-tetrahydro-[1,1′-biphenyl]-2,6-diyl bis(pyrrolidine-3-carboxylate)

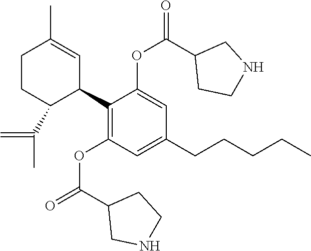 [SMILES: C=C(C)[C@@H]1CCC(C)=C[C@H]1c1c(OC(=O)C2CCNC2)cc(CCCCC)cc1OC(=O)C1CCNC1]

To a solution of (−)-cannabidiol (500 mg, 1.59 mmol) and 1-(tert-butoxycarbonyl)pyrrolidine-3-carboxylic acid (1.03 g, 4.77 mmol) in dry dichloromethane (25 mL) was added 4-(dimethylamino)pyridine (200 mg, 1.63 mmol), and the solution was stirred at ambient temperature for 5 minutes. N,N′-Diisopropylcarbodiimide (600 mg, 4.76 mmol) was added, and the mixture was stirred at ambient temperature overnight. The mixture was concentrated in vacuo to provide 1,1′-di-tent-butyl O′3,O3-((1′R,2′R)-5′-methyl-4-pentyl-2′-(prop-1-en-2-yl)-1′,2′,3′,4′-tetrahydro-[1,1′-biphenyl]-2,6-diyl) bis(pyrrolidine-1,3-dicarboxylate) as a colorless oil (700 mg) that was used in the next step without further purification.

The oil from above (700 mg) was dissolved in dichloromethane (4 mL) and trifluoroacetic acid (2 mL) was added. The solution was stirred at ambient temperature overnight then washed with aqueous sodium bicarbonate until the aqueous layer remained basic. The aqueous layer was extracted with dichloromethane, and the combined organic phases were concentrated in vacuo. The residue was purified by column chromatography, eluting with a gradient of 10% to 30% methanol in dichloromethane to afford the title compound (437 mg, 54% yield).

¹H NMR (600 MHz, DMSO-d₆): δ 6.90 (s, 2H), 4.98 (s, 1H), 4.49 (br s, 1H), 4.31 (br s, 1H), 3.70-3.20 (m, 11H), 2.50 (m, 2H), 2.36 (m, 1H), 2.30-1.90 (m, 6H), 1.70 (m, 2H), 1.59 (s, 3H), 1.52 (m, 2H), 1.52 (s, 3H), 1.32-1.22 (m, 4H), 0.85 (t, J=7.0 Hz, 3H) (two NH not observed); ¹³C NMR (150 MHz, CDCl₃): δ 171.2 (2C), 154.2 (2C), 149.5, 147.7, 142.3, 132.9, 125.9, 121.3, 119.5, 111.1, 48.0, 47.8, 45.5, 45.4, 45.1, 43.5, 42.6, 38.8 (2C), 35.2, 31.5, 30.6, 30.4, 28.9, 28.5, 23.4, 22.4, 20.0, 14.0.

### Synthesis of CBDp13: (1′R,2′R)-6-hydroxy-5′-methyl-4-pentyl-2′-(prop-1-en-2-yl)-1′,2′,3′,4′-tetrahydro-[1,1′-biphenyl]-2-yl 4-methylpiperazine-1-carboxylate

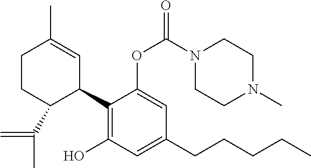 [SMILES: C=C(C)[C@@H]1CCC(C)=C[C@H]1c1c(O)cc(CCCCC)cc1OC(=O)N1CCN(C)CC1]

To a stirred solution of (−)-cannabidiol (200 mg, 0.636 mmol) and 4-methyl-1-piperazinecarbonyl chloride (104 mg, 0.636 mmol) in dichloromethane (5 mL) was added 4-(dimethylamino)pyridine (93 mg, 0.76 mmol) followed by triethylamine (97 mg, 0.96 mmol). The mixture was stirred at ambient temperature overnight then diluted with dichloromethane (25 mL) and washed with water. The organic layer was concentrated in vacuo, and the residue was purified by column chromatography, eluting with a gradient of 30% to 70% ethyl acetate in petroleum ether to afford the title compound as a light amber, viscous oil (142 mg, 51% yield).

¹H NMR (600 MHz, DMSO-d₆): δ 6.72 (s, 1H), 6.40 (s, 1H), 5.33 (br s, 1H), 4.56 (br s, 1H), 4.47 (br s, 1H), 3.73-3.45 (m, 5H), 2.55-2.38 (m, 7H), 2.35 (s, 3H), 2.18 (m, 1H), 2.10-2.01 (m, 1H), 1.85-1.66 (m, 5H), 1.63-1.52 (m, 5H), 1.34-1.24 (m, 4H), 0.87 (t, J=7.0 Hz, 3H) (OH not observed); ¹³C NMR (150 MHz, CDCl₃): δ 155.5, 153.5, 149.6, 147.3, 142.5, 139.7, 123.5, 119.6, 114.2 (2C), 110.9, 54.7, 54.5, 46.1, 45.2, 44.1, 43.6, 37.8, 35.3, 31.5, 30.4, 30.0, 27.7, 23.5, 22.4, 20.2, 13.9.

### Synthesis of CBDp26: (1′R,2′R)-5′-methyl-4-pentyl-2′-(prop-1-en-2-yl)-1′,2′,3′,4′-tetrahydro-[1,1′-biphenyl]-2,6-diyl bis(2-(pyrrolidin-3-yl)acetate)

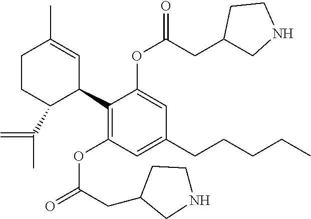 [SMILES: C=C(C)[C@@H]1CCC(C)=C[C@H]1c1c(OC(=O)CC2CCNC2)cc(CCCCC)cc1OC(=O)CC1CCNC1]

4-(Dimethylamino)pyridine (291 mg, 2.38 mmol) was added to a solution of (−)-cannabidiol (500 mg, 1.59 mmol) and 2-(1-(tent-butoxycarbonyOpyrrolidin-3-yl)acetic acid (911 mg, 3.97 mmol) in dry dichloromethane (25 mL), and the solution was stirred at ambient temperature for 5 minutes. N,N′-Diisopropylcarbodiimide (602 mg, 4.77 mmol) was added, and the mixture was stirred at ambient temperature overnight. The mixture was concentrated in vacuo, and the residue was purified by column chromatography eluting with 15% ethyl acetate in petroleum ether to provide di-tent-butyl 3,3′-((((1′R,2′R)-5′-methyl-4-pentyl-2′-(prop-1-en-2-yl)-1′,2′,3′,4′-tetrahydro-[1,1′-biphenyl]-2,6-diyl)bis(oxy))bis(2-oxoethane-2,1-diyl))bis(pyrrolidine-1-carboxylate) as an oil (628 mg).

The oil from above (628 mg) was dissolved in dichloromethane (4 mL) and trifluoroacetic acid (2 mL) was added. After stirring the solution at ambient temperature overnight, an excess of triethylamine (25 mL) was added and the mixture was concentrated in vacuo. The residue was purified by column chromatography, eluting with a gradient of 10% to 40% methanol in dichloromethane to afford the title compound (495 mg, 58% yield).

¹H NMR (500 MHz, DMSO-d₆): δ 6.80 (s, 2H), 4.95 (s, 1H), 4.47 (s, 1H), 4.40 (s, 1H), 3.50-3.20 (m, 7H), 3.20-3.10 (m, 2H) 2.95-2.70 (m, 4H), 2.65-2.45 (m, 5H), 2.25-2.10 (m, 3H), 1.99-1.92 (m, 1H), 1.71-1.61 (m, 4H), 1.59 (s, 3H), 1.53 (m, 2H), 1.53 (s, 3H), 1.33-1.21 (m, 4H), 0.84 (t, J=7.0 Hz, 3H) (two NH not observed); ¹³C NMR (125 MHz, CDCl₃): δ 170.4 (2C), 149.3, 147.4 (2C), 141.6, 132.6, 125.8, 123.8, 110.9, 49.0 (2C), 45.1, 44.3 (2C), 37.6, 36.0, 34.2, 33.6, 33.5, 30.8, 30.0, 29.8 (2C), 29.5 (2C), 28.2, 23.2, 21.9, 19.2, 13.9 (two aromatic C not observed).

### Synthesis of CBDp27: (1′R,2′R)-6-hydroxy-5′-methyl-4-pentyl-2′-(prop-1-en-2-yl)-1′,2′,3′,4′-tetrahydro-[1,1′-biphenyl]-2-yl (2-(dimethylamino)ethyl)carbamate

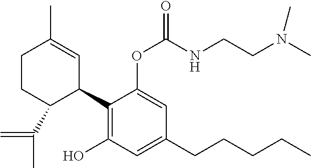 [SMILES: C=C(C)[C@@H]1CCC(C)=C[C@H]1c1c(O)cc(CCCCC)cc1OC(=O)NCCN(C)C]

A solution of N,N-dimethylethylenediamine (5.98 g, 67.8 mmol) in toluene (5 mL) was added dropwise to a suspension of 1,1′-carbonyldiimidazole (13.20 g, 81.41 mmol) in toluene (29 mL) at 0° C. under nitrogen atmosphere. The mixture was stirred at 0° C. for 1 hour then at ambient temperature for 17 hours. The solution was concentrated in vacuo to provide a yellow oil that was used in the next step without further purification.

A solution of the yellow oil from above and (−)-cannabidiol (5.345 g, 17.00 mmol) in dichloromethane (42 mL) was stirred at ambient temperature while triethylamine (4.7 mL, 34 mmol) was added. The solution was stirred at ambient temperature for 5 days under nitrogen atmosphere then concentrated in vacuo followed by azeotropic removal of triethylamine using ethyl acetate (3×40 mL). The residue was dissolved in ethyl acetate (200 mL) and washed with water (3×80 mL) then dried over anhydrous sodium sulfate, filtered and concentrated in vacuo. The residue was purified by column chromatography, eluting with 15% ethyl acetate in hexanes followed by a gradient of 15% to 25% ethyl acetate (containing 10% triethylamine and 10% isopropanol) in hexanes to afford the title compound as a pale, sticky oil (3.57 g, 49% yield).

¹H NMR (500 MHz, CDCl₃): δ 6.50 (br s, 1H), 6.46 (s, 1H), 5.52 (m, 2H), 4.57 (br s, 1H), 4.23 (br s, 1H), 3.62 (m, 1H), 3.32 (m, 2H), 2.50-2.43 (m, 5H), 2.26 (s, 6H), 2.24-2.17 (m, 1H), 2.09-2.02 (m, 1H), 1.82-1.69 (m, 2H), 1.76 (s, 3H), 1.62 (s, 3H), 1.57 (m, 2H), 1.33-1.24 (m, 4H), 0.87 (t, J=6.9 Hz, 3H) (NH not observed); ¹³C NMR (125 MHz, CDCl₃): δ 155.4, 154.6, 149.5, 147.1, 142.6, 139.8, 123.7, 119.5, 114.2 (2C), 111.3, 58.1 (2C), 45.7, 45.1, 38.4, 37.4, 35.3, 31.4, 30.5, 30.2, 28.0, 23.5, 22.4, 19.4, 14.0.

### Alternate Method for the Synthesis of CBDp27

Triphosgene (70 mg, 0.24 mmol) was added to a solution of (−)-cannabidiol (250 mg, 0.794 mmol) and triethylamine (96 mg, 0.95 mmol) in dichloromethane at 0° C. The mixture was stirred at 0° C. for 15 minutes then at ambient temperature for 3 hours. Saturated aqueous sodium bicarbonate was added, and the aqueous phase was extracted with dichloromethane (50 mL). The combined organic layers were dried over anhydrous sodium sulfate, filtered and concentrated in vacuo to provide (1′R,2′R)-6-hydroxy-5′-methyl-4-pentyl-2′-(prop-1-en-2-yl)-1′,2′,3′,4′-tetrahydro-[1,1′-biphenyl]-2-yl carbonochloridate as an oil (276 mg) that was used in the next step without further purification.

N,N-dimethylethylenediamine (65 mg, 0.74 mmol) was added to a stirred solution of the oil from above (276 mg, 0.732 mmol) in dichloromethane, and the mixture was stirred at ambient temperature overnight. The mixture was concentrated in vacuo, and the residue was purified by column chromatography, eluting with 10% methanol in dichloromethane to afford the title compound as a colourless oil (156 mg, 46% yield).

### Synthesis of CBDp17: (1′R,2′R)-6-hydroxy-5′-methyl-4-pentyl-2′-(prop-1-en-2-yl)-1′,2′,3′,4′-tetrahydro-[1,1′-biphenyl]-2-yl piperidine-1-carboxylate

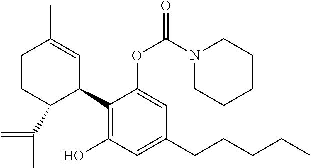 [SMILES: C=C(C)[C@@H]1CCC(C)=C[C@H]1c1c(O)cc(CCCCC)cc1OC(=O)N1CCCCC1]

(−)-Cannabidiol (200 mg, 0.636 mmol) and 4-(dimethylamino)pyridine (78 mg, 0.64 mmol) were added to a stirred solution of piperidine-1-carbonyl chloride (94 mg, 0.64 mmol) in dichloromethane (5 mL) followed by the addition of triethylamine (65 mg, 0.64 mmol). The mixture was stirred at ambient temperature for 4 hours then diluted with dichloromethane (25 mL) and washed with saturated aqueous sodium bicarbonate and water. The organic layer was dried over anhydrous magnesium sulfate, filtered and concentrated in vacuo. The residue was purified by column chromatography, eluting with a gradient of 10% to 40% ethyl acetate in petroleum ether to afford the title compound as a pale solid (119 mg, 44% yield).

¹H NMR (600 MHz, CDCl₃): δ 6.52 (br s, 1H), 6.42 (s, 1H), 6.03 (br s, 1H), 5.60 (br s, 1H), 4.61 (br s, 1H), 4.46 (br s, 1H), 3.60-3.50 (m, 4H), 3.45 (br s, 1H), 2.48 (t, J=7.9 Hz, 2H), 2.45 (m, 1H), 2.20 (m, 1H), 2.07 (m, 1H), 1.82 (m, 1H), 1.77-1.70 (m, 1H), 1.76 (br s, 3H), 1.67-1.55 (m, 6H), 1.59 (s, 3H), 1.57 (m, 2H), 1.34-1.24 (m, 4H), 0.87 (t, J=6.9 Hz, 3H); ¹³C NMR (150 MHz, CDCl₃): δ 155.5, 153.6, 149.9, 147.3, 142.6, 140.0, 123.7, 119.6, 114.4, 114.1, 111.0, 45.4 (2C), 45.0, 37.8, 35.4, 31.5, 30.5, 30.1, 27.7, 25.5 (2C), 24.3, 23.6, 22.5, 20.2, 14.0.

### Synthesis of CBDp18: (1′R,2′R)-5′-methyl-4-pentyl-2′-(prop-1-en-2-yl)-1′,2′,3′,4′-tetrahydro-[1,1′-biphenyl]-2,6-diyl bis(piperidine-1-carboxylate)

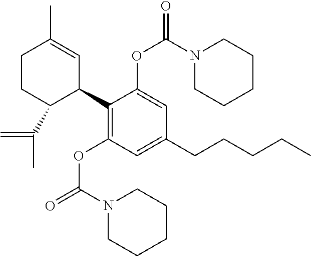 [SMILES: C=C(C)[C@@H]1CCC(C)=C[C@H]1c1c(OC(=O)N2CCCCC2)cc(CCCCC)cc1OC(=O)N1CCCCC1]

To a stirred solution of (−)-cannabidiol (250 mg, 0.794 mmol) and piperidine-1-carbonyl chloride (300 mg, 2.03 mmol) in dichloromethane (5 mL) was added 4-(dimethylamino)pyridine (150 mg, 1.23 mmol) followed by triethylamine (250 mg, 2.47 mmol). The mixture was stirred at ambient temperature overnight then diluted with dichloromethane (25 mL) and washed with saturated aqueous sodium bicarbonate and water. The organic layer was dried over anhydrous magnesium sulfate, filtered and concentrated in vacuo. The residue was purified by column chromatography, eluting with a gradient of 10% to 30% ethyl acetate in petroleum ether to afford the title compound as a viscous solid (349 mg, 82% yield).

¹H NMR (500 MHz, CDCl_{3,}): δ 6.70 (s, 2H), 5.34 (s, 1H), 4.56 (s, 1H), 4.50 (s, 1H), 3.66-3.34 (m, 9H), 2.52 (t, J=7.9 Hz, 2H), 2.50 (m, 1H), 2.16-1.97 (m, 2H), 1.88-1.52 (m, 22H), 1.34-1.26 (m, 4H), 0.87 (t, J=6.9 Hz, 3H); ¹³C NMR (125 MHz, CDCl₃): δ 153.4 (2C), 150.2 (2C), 147.9, 141.5, 131.7, 126.6, 125.1, 120.5, 120.0, 110.8, 49.9 (2C), 47.5 (2C), 46.1, 38.4, 35.3, 31.7, 30.7, 30.5, 29.0, 25.9 (2C), 25.4 (2C), 24.1 (2C), 23.3, 22.5, 20.0, 14.0.

### Synthesis of CBGp1: (E)-2-(3,7-dimethylocta-2,6-dien-1-yl)-3-hydroxy-5-pentylphenyl pyrrolidine-3-carboxylate

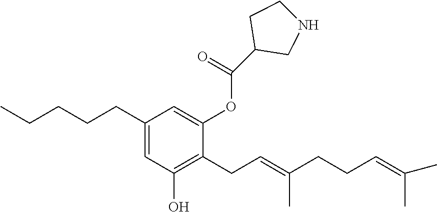 [SMILES: CCCCCc1cc(O)c(C/C=C(\C)CCC=C(C)C)c(OC(=O)C2CCNC2)c1]

A solution of cannabigerol (6.395 g, 20.21 mmol) and 1-(9H-fluoren-9-ylmethoxycarbonyl)pyrrolidine-3-carboxylic acid (5.11 g, 15.1 mmol) in dry dichloromethane (100 mL) was stirred at ambient temperature while 4-(dimethylamino)pyridine (2.47 g, 20.2 mmol) was added followed by N,N′-diisopropylcarbodiimide (3.13 mL, 20.2 mmol). The mixture was stirred at ambient temperature in the dark under nitrogen atmosphere overnight then concentrated in vacuo. The residue was dissolved in ethyl acetate (250 mL) and washed with saturated aqueous ammonium chloride (2×90 mL) and brine (50 mL). The organic layer was dried over anhydrous sodium sulfate, filtered and concentrated in vacuo. The residue was purified by column chromatography, eluting with a gradient of 10% to 20% ethyl acetate in hexanes to provide (E)-1-((9H-fluoren-9-yl)methyl) 3-(2-(3,7-dimethylocta-2,6-dien-1-yl)-3-hydroxy-5-pentylphenyl) pyrrolidine-1,3-dicarboxylate as a yellow oil (4.8 g, 50% yield).

The oil from above (4.8 g) was combined with additional batches of (E)-1-((9H-fluoren-9-yl)methyl) 3-(2-(3,7-dimethylocta-2,6-dien-1-yl)-3-hydroxy-5-pentylphenyl) pyrrolidine-1,3-dicarboxylate (13.3 g) that were prepared in the same manner as described above. (E)-1-((9H-fluoren-9-yl)methyl) 3-(2-(3,7-dimethylocta-2,6-dien-1-yl)-3-hydroxy-5-pentylphenyl) pyrrolidine-1,3-dicarboxylate (18.1 g, 28.5 mmol) was dissolved in dry N,N-dimethylformamide (80 mL) and piperidine (20 mL) was added. The solution was stirred at ambient temperature in the dark under nitrogen atmosphere for 4 hours then diluted with ethyl acetate (600 mL) and washed with brine (7×100 mL). The organic layer was dried over anhydrous sodium sulfate, filtered and concentrated in vacuo. The residue was purified by column chromatography, eluting with a gradient of 1% to 10% methanol in dichloromethane to afford the title compound (7.7 g, 65% yield).

¹H NMR (600 MHz, CDCl₃): δ 6.53 (s, 1H), 6.41 (s, 1H), 5.22 (m, 1H), 5.08 (m, 1H), 4.59 (br s, 1H), 3.40-2.96 (m, 5H), 3.21 (d, J=6.7 Hz, 2H), 2.50 (t, J=7.8 Hz, 2H), 2.17 (m, 2H), 2.10-1.99 (m, 4H), 1.76 (s, 3H), 1.66 (s, 3H), 1.58 (s, 3H), 1.57-1.54 (m, 2H), 1.32-1.28 (m, 4H), 0.88 (t, J=6.9 Hz, 3H) (NH not observed); ¹³C NMR (150 MHz, CDCl₃): δ 173.9, 155.7, 149.1, 142.5, 138.1, 131.9, 123.8, 121.3, 117.0, 113.9, 113.8, 50.9, 47.3, 43.9, 39.6, 35.4, 31.5, 30.7, 29.7, 26.4, 25.6, 23.2, 22.5, 17.7, 16.2, 14.0.

### Alternate Method for the Synthesis of CBGp1

4-(Dimethylamino)pyridine (150 mg, 1.22 mmol) was added to a solution of cannabigerol (500 mg, 1.58 mmol) and 1-(9H-fluoren-9-ylmethoxycarbonyl)pyrrolidine-3-carboxylic acid (400 mg, 1.18 mmol) in dry dichloromethane (30 mL), and the solution was stirred at ambient temperature for 5 minutes. N,N′-Diisopropylcarbodiimide (200 mg, 1.58 mmol) was added, and the mixture was stirred at ambient temperature overnight. The mixture was concentrated in vacuo, and the residue was purified by column chromatography, eluting with 10% ethyl acetate in petroleum ether to provide (E)-1-((9H-fluoren-9-yl)methyl) 3-(2-(3,7-dimethylocta-2,6-dien-1-yl)-3-hydroxy-5-pentylphenyl) pyrrolidine-1,3-dicarboxylate as an oil (480 mg).

The oil from above (480 mg) was dissolved in dichloromethane (10 mL) and piperidine (10 mL) was added. The solution was stirred at ambient temperature overnight, and the mixture was concentrated in vacuo. The residue was purified by column chromatography, eluting with a gradient of 10% to 50% methanol in dichloromethane to afford the title compound (235 mg, 36% yield).

### Synthesis of CBGp2: (E)-2-(3,7-dimethylocta-2,6-dien-1-yl)-3-hydroxy-5-pentylphenyl 4-methylpiperazine-1-carboxylate

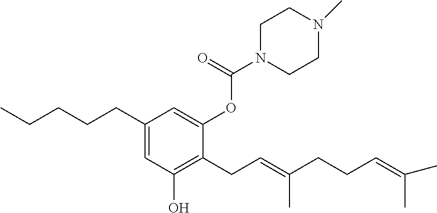 [SMILES: CCCCCc1cc(O)c(C/C=C(\C)CCC=C(C)C)c(OC(=O)N2CCN(C)CC2)c1]

To a stirred solution of cannabigerol (250 mg, 0.789 mmol) and 4-methyl-1-piperazinecarbonyl chloride (103 mg, 0.631 mmol) in dichloromethane (5 mL) was added 4-(dimethylamino)pyridine (75 mg, 0.61 mmol) followed by triethylamine (125 mg, 1.24 mmol). The mixture was stirred at ambient temperature overnight then diluted with dichloromethane (40 mL) and washed with water. The organic layer was concentrated in vacuo, and the residue was purified by column chromatography, eluting with 10% methanol in dichloromethane to afford the title compound as a light amber, viscous oil (248 mg, 71% yield).

¹H NMR (500 MHz, CDCl₃): δ 6.48 (s, 2H), 5.80 (br s, 1H), 5.22 (m, 1H), 5.08 (m, 1H), 3.69 (br s, 2H), 3.59 (br s, 2H), 3.24 (d, J=6.9 Hz, 2H), 2.50-2.44 (m, 6H), 2.34 (s, 3H), 2.11-1.99 (m, 4H), 1.77 (s, 3H), 1.66 (s, 3H), 1.58 (s, 3H), 1.57-1.54 (m, 2H) 1.33-1.28 (m, 4H), 0.88 (t, J=6.9 Hz, 3H); ¹³C NMR (125 MHz, CDCl₃): δ 155.2, 153.6, 149.6, 142.3, 137.8, 131.8, 123.9, 121.7, 117.3, 114.6, 113.4, 54.8, 54.7, 46.2, 44.4, 43.8, 39.6, 35.5, 31.5, 30.7, 26.4, 25.6, 23.2, 22.5, 17.7, 16.2, 14.0.

### Synthesis of CBGp3: (E)-2-(3,7-dimethylocta-2,6-dien-1-yl)-5-pentyl-1,3-phenylene bis(4-methylpiperazine-1-carboxylate)

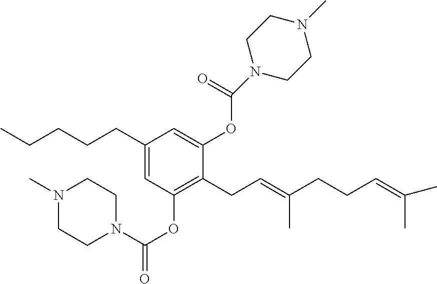 [SMILES: CCCCCc1cc(OC(=O)N2CCN(C)CC2)c(C/C=C(\C)CCC=C(C)C)c(OC(=O)N2CCN(C)CC2)c1]

To a stirred solution of cannabigerol (250 mg, 0.789 mmol) and 4-methyl-1-piperazinecarbonyl chloride (322 mg, 1.98 mmol) in dichloromethane (5 mL) was added 4-(dimethylamino)pyridine (100 mg, 0.818 mmol) followed by triethylamine (240 mg, 2.37 mmol). The mixture was stirred at ambient temperature overnight then diluted with dichloromethane (50 mL) and washed with water. The organic layer was concentrated in vacuo, and the residue was purified by column chromatography, eluting with 10% methanol in dichloromethane to afford the title compound as a light amber, viscous oil (296 mg, 66% yield).

¹H NMR (600 MHz, CDCl₃): δ 6.80 (s, 2H), 5.11 (m, 1H), 5.06 (m, 1H), 3.67 (br s, 4H), 3.58 (br s, 4H), 3.18 (d, J=6.5 Hz, 2H), 2.55 (t, J=7.9, 2H), 2.43 (m, 8H), 2.33 (s, 6H), 2.02 (m, 2H), 1.94 (m, 2H), 1.67 (s, 3H), 1.65 (s, 3H), 1.60 (m, 2H), 1.57 (s, 3H), 1.31-1.29 (m, 4H), 0.88 (t, J=6.9 Hz, 3H); ¹³C NMR (150 MHz, CDCl₃) δ: 153.3 (2C), 149.8 (2C), 141.8, 135.0, 131.3, 124.1, 123.9, 121.7, 119.8 (2C), 54.8 (2C), 54.6 (2C), 46.4 (2C), 44.4 (2C), 43.9 (2C), 39.6, 35.8, 31.5, 30.6, 26.6, 25.6, 23.5, 22.4, 17.7, 16.3, 14.0.

### Synthesis of CBGp5: (E)-2-(3,7-dimethylocta-2,6-dien-1-yl)-3-hydroxy-5-pentylphenyl piperazine-1-carboxylate

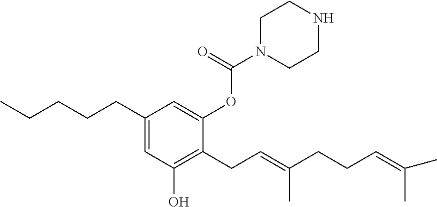 [SMILES: CCCCCc1cc(O)c(C/C=C(\C)CCC=C(C)C)c(OC(=O)N2CCNCC2)c1]

Triphosgene (118 mg, 0.397 mmol) was added to a solution of cannabigerol (250 mg, 0.789 mmol) and triethylamine (125 mg, 1.23 mmol) in dichloromethane at 0° C. The mixture was stirred at 0° C. for 15 minutes then at ambient temperature for 3 hours. Saturated aqueous sodium bicarbonate was added, and the aqueous phase was extracted with dichloromethane (50 mL). The combined organic layers were dried over anhydrous sodium sulfate, filtered and concentrated in vacuo to provide (E)-2-(3,7-dimethylocta-2,6-dien-1-yl)-3-hydroxy-5-pentylphenyl carbonochloridate as an oil (175 mg) that was used in the next step without further purification.

Piperazine (75 mg, 0.87 mmol) was added to a stirred solution of the oil from above (175 mg, 0.461 mmol) in dichloromethane, and the mixture was stirred at ambient temperature overnight. The mixture was concentrated in vacuo, and the residue was purified by column chromatography, eluting with 10% methanol in dichloromethane to afford the title compound as a light pink solid (186 mg, 55% yield).

¹H NMR (500 MHz, CDCl₃): δ 6.48 (s, 1H), 6.46 (s, 1H), 5.22 (t, J=6.9 Hz, 1H), 5.05 (t, J=6.9 Hz, 1H), 3.65 (br s, 2H), 3.55 (br s, 2H), 3.24 (d, J=6.9 Hz, 2H), 2.92 (m, 4H), 2.48 (t, J=7.8, 2H), 2.10-2.05 (m, 2H), 2.02-1.99 (m, 2H), 1.76 (s, 3H), 1.66 (s, 3H), 1.58 (s, 3H), 1.55-1.53 (m, 2H) 1.33-1.27 (m, 4H), 0.87 (t, J=6.9 Hz, 3H) (NH and OH not observed); ¹³C NMR (125 MHz, CDCl₃) δ: 155.4, 153.8, 149.7, 142.2, 137.3, 131.7, 123.9, 121.8, 117.6, 114.4, 113.4, 45.8, 45.7, 45.4, 44.8, 39.6, 35.5, 31.5, 30.7, 26.5, 25.6, 23.2, 22.5, 17.6, 16.2, 14.0.

### Synthesis of CBCp1: 2-methyl-2-(4-methylpent-3-en-1-yl)-7-pentyl-2H-chromen-5-yl4-methylpiperazine-1-carboxylate

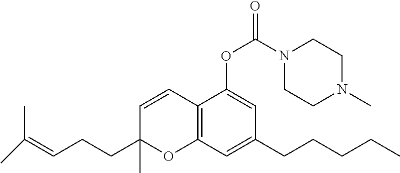 [SMILES: CCCCCc1cc(OC(=O)N2CCN(C)CC2)c2c(c1)OC(C)(CCC=C(C)C)C=C2]

To a stirred solution of cannabichromene (250 mg, 0.795 mmol) and 4-methyl-1-piperazinecarbonyl chloride (194 mg, 1.19 mmol) in dichloromethane (10 mL) was added 4-(dimethylamino)pyridine (100 mg, 0.818 mmol) followed by triethylamine (160 mg, 1.58 mmol). The mixture was stirred at ambient temperature overnight then diluted with dichloromethane (50 mL) and washed with water. The organic layer was concentrated in vacuo, and the residue was purified by column chromatography, eluting with 10% methanol in dichloromethane to afford the title compound as a viscous oil (221 mg, 63% yield).

¹H NMR (500 MHz, CDCl₃): δ 6.49 (s, 1H), 6.46 (s, 1H), 6.36 (d, J=10.1 Hz, 1H), 5.52 (d, J=10.1 Hz, 1H), 5.10 (t, J=7.1 Hz, 1H), 3.70 (br s, 2H), 3.50 (br s, 2H), 2.49 (t, J=7.8 Hz, 2H), 2.44 (m, 4H), 2.33 (s, 3H), 2.10 (m, 2H), 1.75-1.66 (m, 2H), 1.68 (s, 3H), 1.61-1.55 (m, 2H), 1.57 (s, 3H), 1.37 (s, 3H), 1.35-1.25 (m, 4H), 0.88 (t, J=6.7 Hz, 3H); ¹³C NMR (100 MHz, CDCl₃): δ 153.5, 153.2, 146.5, 144.1, 131.4, 128.8, 124.0, 116.7, 114.1, 113.3, 111.8, 78.2, 54.7, 54.5, 46.0, 44.3, 44.8, 41.0, 35.7, 31.4, 30.4, 26.2, 25.5, 22.6, 22.4, 17.5, 13.9.

### Synthesis of CBCp3: 2-methyl-2-(4-methylpent-3-en-1-yl)-7-pentyl-2H-chromen-5-yl piperazine-1-carboxylate

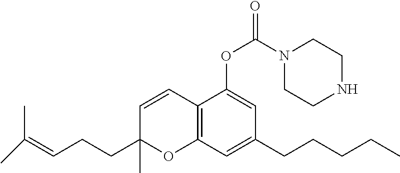 [SMILES: CCCCCc1cc(OC(=O)N2CCNCC2)c2c(c1)OC(C)(CCC=C(C)C)C=C2]

Triphosgene (150 mg, 0.505 mmol) was added to a solution of cannabichromene (500 mg, 1.59 mmol) and triethylamine (161 mg, 1.59 mmol) in dichloromethane at 0° C. The mixture was stirred at 0° C. for 15 minutes then at ambient temperature for 3 hours. Saturated aqueous sodium bicarbonate was added, and the aqueous phase was extracted with dichloromethane (50 mL). The combined organic layers were dried over anhydrous sodium sulfate, filtered and concentrated in vacuo to provide 2-methyl-2-(4-methylpent-3-en-1-yl)-7-pentyl-2H-chromen-5-yl carbonochloridate as an oil (300 mg) that was used in the next step without further purification.

Piperazine (137 mg, 1.59 mmol) was added to a stirred solution of the oil from above (300 mg, 0.795 mmol) in dichloromethane, and the mixture was stirred at ambient temperature overnight. The mixture was concentrated in vacuo, and the residue was purified by column chromatography, eluting with a gradient of 10% to 30% methanol in dichloromethane to afford the title compound as a light pink solid (380 mg, 56% yield).

¹H NMR (500 MHz, CDCl₃): δ 6.49 (s, 1H), 6.47 (s, 1H), 6.37 (d, J=10.1 Hz, 1H), 5.53 (d, J=10.0 Hz, 1H), 5.09 (t, J=7.1 Hz, 1H), 3.65 (br s, 2H), 3.55 (br s, 2H), 2.92 (m, 4H), 2.49 (t, J=7.8 Hz, 2H), 2.10 (m, 2H), 2.03 (br s, 1H), 1.76-1.66 (m, 2H), 1.66 (s, 3H), 1.61-1.55 (m, 2H), 1.56 (s, 3H), 1.38 (s, 3H), 1.33-1.28 (m, 4H), 0.88 (t, J=6.9 Hz, 3H); ¹³C NMR (125 MHz, CDCl₃): δ 153.6, 153.4, 146.7, 144.3, 131.6, 129.0, 124.0, 116.8, 114.2, 113.4, 111.9, 78.3, 45.9, 45.8, 45.6, 44.9, 41.1, 35.8, 31.5, 30.5, 26.3, 25.6, 22.7, 22.5, 17.6, 14.0.

### Synthesis of THCp1: (6aR,10aR)-6,6,9-trimethyl-3-pentyl-6a,7,8,10a-tetrahydro-6H-benzo[c]chromen-1-yl 4-methylpiperazine-1-carboxylate

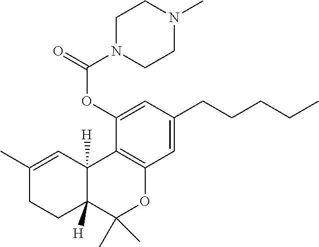 [SMILES: CCCCCc1cc(OC(=O)N2CCN(C)CC2)c2c(c1)OC(C)(C)[C@@H]1CCC(C)=C[C@@H]21]

To a stirred solution of (−)-trans-Δ⁹-tetrahydrocannabinol (250 mg, 0.795 mmol) and 4-methyl-1-piperazinecarbonyl chloride (325 mg, 2.00 mmol) in dichloromethane (5 mL) was added 4-(dimethylamino)pyridine (100 mg, 0.818 mmol) followed by triethylamine (240 mg, 2.37 mmol). The mixture was stirred at ambient temperature overnight then diluted with dichloromethane and washed with water. The organic layer was concentrated in vacuo, and the residue was purified by column chromatography, eluting with a gradient of 0% to 10% methanol in dichloromethane to afford the title compound as a light amber, viscous oil (180 mg, 51% yield).

¹H NMR (600 MHz, CDCl₃): δ 6.53 (s, 1H), 6.46 (s, 1H), 5.95 (s, 1H), 3.74-3.56 (m, 4H), 3.18 (m, 1H), 2.48 (t, J=7.2 Hz, 2H), 2.44 (m, 4H), 2.33 (s, 3H), 2.14 (m, 2H), 1.89 (m, 1H), 1.69 (m, 1H), 1.65 (s, 3H), 1.57 (m, 2H), 1.40 (s, 3H), 1.39 (m, 1H), 1.33-1.27 (m, 4H), 1.10 (s, 3H), 0.87 (t, J=7.0 Hz, 3H); ¹³C NMR (150 MHz, CDCl₃): δ 154.3, 152.8, 149.7, 142.5, 134.1, 123.7, 115.2, 114.8, 114.4, 77.3, 54.8 (2C), 46.2, 45.5, 44.5, 43.9, 35.4, 33.9, 31.5, 30.9, 30.5, 27.4, 24.8, 23.5, 22.5, 19.4, 14.0.

### Synthesis of THCp2: (6aR,10aR)-6,6,9-trimethyl-3-pentyl-6a,7,8,10a-tetrahydro-6H-benzo[c]chromen-1-yl pyrrolidine-3-carboxylate

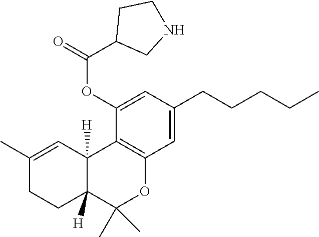 [SMILES: CCCCCc1cc(OC(=O)C2CCNC2)c2c(c1)OC(C)(C)[C@@H]1CCC(C)=C[C@@H]21]

4-(Dimethylamino)pyridine (19 mg, 0.15 mmol) was added to a solution of (−)-trans-Δ⁹-tetrahydrocannabinol (480 mg, 1.53 mmol), 1-(9H-fluoren-9-ylmethoxycarbonyl)pyrrolidine-3-carboxylic acid (570 mg, 1.68 mmol) and 1-ethyl-3-(3-dimethylaminopropyl)carbodiimide (320 mg, 1.68 mmol) in dichloromethane (10 mL), and the mixture was stirred at ambient temperature overnight. The mixture was diluted with dichloromethane (30 mL) then washed with 0.5 M hydrochloric acid and water. The organic phase was dried over anhydrous sodium sulfate, filtered and concentrated in vacuo. The residue was purified by column chromatography, eluting with a gradient of 0% to 50% ethyl acetate in petroleum ether to provide 1-((9H-fluoren-9-yl)methyl) 3-((6aR,10aR)-6,6,9-trimethyl-3-pentyl-6a,7,8,10a-tetrahydro-6H-benzo[c]chromen-1-yl) pyrrolidine-1,3-dicarboxylate as a colourless solid (800 mg, 83% yield).

The colourless solid from above (285 mg, 0.450 mmol) was dissolved in dichloromethane (8 mL) and piperidine (2 mL) was added. The solution was stirred at ambient temperature for 1 hour then concentrated in vacuo. The residue was dissolved in ethyl acetate and washed with aqueous citric acid, saturated aqueous sodium bicarbonate and water. The organic phase was dried over anhydrous sodium sulfate then filtered and concentrated in vacuo. The residue was dissolved in diethyl ether (5 mL) and treated with oxalic acid (45 mg, 0.50 mmol) then crystallized from diethyl ether in hexane to afford (6aR,10aR)-6,6,9-trimethyl-3-pentyl-6a,7,8,10a-tetrahydro-6H-benzo[c] chromen-1-yl pyrrolidine-3-carboxylate as the oxalic acid salt as a light yellow solid (140 mg, 62% yield).

¹H NMR (600 MHz, CDCl₃): δ6.52 (s, 1H), 6.47 (s, 1H), 5.91 (s, 0.5H), 5.89 (s, 0.5H), 3.72-3.58 (m, 3H), 3.47-3.40 (m, 2H), 3.03 (m, 1H), 2.50 (t, J=7.7 Hz, 2H), 2.47-2.32 (m, 2H), 2.17 (m, 2H), 1.98-1.93 (m, 1H), 1.69 (s, 3H), 1.64-1.55 (m, 3H), 1.40 (s, 3H), 1.39-1.26 (m, 5H), 1.05 (s, 3H), 0.90 (t, J=7.1 Hz, 3H) (NH and OH not observed); ¹³C NMR (150 MHz, CDCl₃): δ 171.8, 164.0 (2C), 156.0, 150.7, 144.3, 136.8 (0.5C), 136.7 (0.5C), 124.8 (0.5C), 124.5 (0.5C), 116.7, 116.7 (0.5C), 116.5 (0.5C), 115.0 (0.5C), 114.9 (0.5C), 78.7, 48.3, 47.3, 46.7, 43.5, 35.4, 35.5, 32.9, 32.7, 32.1, 32.0, 29.3, 26.1, 23.8, 23.7, 19.5, 14.5.

### Synthesis of THCp3: (6aR,10aR)-6,6,9-trimethyl-3-pentyl-6a,7,8,10a-tetrahydro-6H-benzo[c]chromen-1-yl piperazine-1-carboxylate

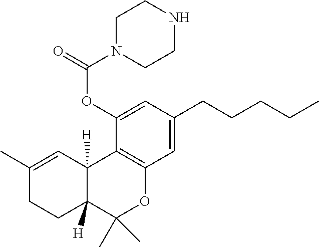 [SMILES: CCCCCc1cc(OC(=O)N2CCNCC2)c2c(c1)OC(C)(C)[C@@H]1CCC(C)=C[C@@H]21]

Triphosgene (100 mg, 0.337 mmol) was added to a solution of (−)-trans-Δ⁹-tetrahydrocannabinol (210 mg, 0.668 mmol) and triethylamine (101 mg, 1.00 mmol) in dichloromethane (5 mL) at 0° C. The mixture was stirred at 0° C. for 15 minutes then at ambient temperature for 16 hours. The mixture was washed with water then dried over anhydrous sodium sulfate, filtered and concentrated in vacuo to provide (6aR,10aR)-6,6,9-trimethyl-3-pentyl-6a,7,8,10a-tetrahydro-6H-benzo[c] chromen-1-yl carbonochloridate as an oil (250 mg) that was used in the next step without further purification.

Piperazine (288 mg, 3.34 mmol) was added to a stirred solution of the oil from above (250 mg, 0.663 mmol) in dichloromethane, and the mixture was stirred at ambient temperature overnight. The mixture was concentrated in vacuo, and the residue was purified by column chromatography, eluting with a gradient of 0% to 10% methanol in dichloromethane to afford the title compound as a light pink solid (108 mg, 38% yield).

¹H NMR (600 MHz, CDCl₃): δ 6.52 (s, 1H), 6.44 (s, 1H), 5.96 (s, 1H), 3.77-3.44 (m, 4H), 3.18 (m, 1H), 2.96-2.89 (m, 4H), 2.48 (t, J=7.8 Hz, 2H), 2.14 (m, 2H), 1.89 (m, 1H), 1.69 (m, 1H), 1.65 (s, 3H), 1.55 (m, 2H), 1.40 (s, 3H), 1.38 (m, 1H), 1.30-1.28 (m, 4H), 1.09 (s, 3H), 0.87 (t, J=7.0 Hz, 3H) (NH not observed); ¹³C NMR (150 MHz, CDCl₃): δ 154.3, 152.9, 149.7, 142.5, 134.1, 123.7, 115.2, 114.8, 114.4, 77.3, 45.9 (2C), 45.7, 45.5, 45.0, 35.4, 33.9, 31.5, 30.9, 30.5, 27.4, 24.8, 23.4, 22.5, 19.4, 14.0.

### Example 2: High-Performance Liquid Chromatography (HPLC) Methods

### LC/MS/MS Instrumentation

The HPLC system consisted of an Agilent 1200 Series Rapid Resolution LC System (Agilent Technologies, Waldbronn, Germany) with a solvent micro vacuum degasser, a binary pump, a thermostatted column compartment, and an autosampler. The mass analysis was made with an Agilent 6410 Triple Quadrupole LC/MS equipped with an electrospray ionization source (Agilent Technologies, Palo Alto, Calif., USA). Data were acquired by Agilent MassHunter Workstation Acquisition software (Agilent Technologies, Data Acquisition for Triple Quad., version B.01.03).

### HPLC Method

Agilent 1100 Series: G1316A Concom, G1313A ALS, G1315B DAD, G1312A BinPump, G1322A Degasser; Zorbax Eclipse Plus C₁₈ column, 3.0×100 mm, 3.5 micron; 210 nm. Gradient method: A solvent—0.1% formic acid in H₂O; B solvent—0.1% formic acid in acetonitrile; 0-3 min—30% to 85% B; 3-7 min—85% B; 7-7.1 min—85% to 30% B; 7.1-10 min—30% B.

For LC-MS analysis, five microliters of the sample solution were injected onto a reversed-phase HPLC column (Zorbax Extend-C₁₈ Rapid Resolution HT 2.1×50 mm, 1.8 μm) (Agilent Technologies, Palo Alto, Calif, USA). The column temperature was 60° C., flow rate 0.4 ml/min, and gradient elution was used with water (eluent A) and methanol (eluent B) both containing 0.1% (v/v) of formic acid. Following gradient profile was employed: 0-1.5 min: 35→95% B; 1.5-4.5 min: 95% B; 4.5→4.51: 95→35% B; and 4.51-7.0 min: 35% B. An autosampler tray temperature was set at 10° C. The following ionization conditions were used: ESI positive ion mode, drying gas (nitrogen) temperature 300° C., drying gas flow rate 7.5 l/min, nebulizer pressure 35 psi, and capillary voltage 4000 V. Analyte detection was performed using multiple reaction monitoring (MRM) with a dwell time of 10 ms to each transition. Mass resolution for MS1 and MS2 quadrupoles were 0.7 FWHM and 1.2 FWHM, respectively.

### Results

MRM transitions (m/z), fragmentor voltage (V), collision energy (V), and mass resolution (FWHM) values for CBG, CBC, CBD, THC, and various prodrugs are presented in Table 1 below. Weighted least-squares linear regression was used for obtaining calibration curves with Agilent MassHunter software (Quantitative Analysis Version B.09.00). Concentrations were calculated with external standard method.

TABLE 1

| | Precursor ion | Product ion | Fragmentor | Collision |
|---|---|---|---|---|
| Compound | (m/z) | (m/z) | voltage (V) | energy (V) |
| CBGp3 | 569.3 | 445 | 200 | 16 |
| CBGp3 | 569.3 | 433 | 200 | 16 |
| CBGp2 | 443.4 | 319 | 150 | 12 |
| CBGp2 | 443.4 | 307 | 150 | 12 |
| CBCp1 | 441.3 | 319 | 150 | 16 |
| CBCp1 | 441.3 | 127 | 150 | 16 |
| THCp1 | 441.2 | 347 | 175 | 20 |
| THCp1 | 441.2 | 307 | 175 | 20 |
| CBDp27 | 429.3 | 315 | 175 | 18 |
| CBDp27 | 429.3 | 115 | 175 | 18 |
| CBGp5 | 429.3 | 305 | 150 | 8 |
| CBGp5 | 429.3 | 293 | 150 | 8 |
| CBCp3 | 427.3 | 345 | 150 | 20 |
| CBCp3 | 427.3 | 305 | 150 | 20 |
| THCp3 | 427.3 | 315 | 175 | 15 |
| THCp3 | 427.3 | 293 | 175 | 15 |
| CBCp3 | 427.3 | 113 | 150 | 20 |
| CBGp1 | 414.3 | 290 | 160 | 20 |
| CBGp1 | 414.3 | 193 | 160 | 20 |
| CBG | 317.2 | 193 | 150 | 12 |
| CBG | 317.2 | 123 | 150 | 12 |
| CBC | 315.3 | 259 | 150 | 16 |
| CBC | 315.3 | 233 | 150 | 16 |
| CBC | 315.3 | 193 | 150 | 16 |
| CBD | 315.2 | 259 | 175 | 19 |
| CBD | 315.2 | 193 | 175 | 19 |
| THC | 315.2 | 259 | 120 | 20 |
| THC | 315.2 | 193 | 120 | 20 |

### Example 3: Stability in Aqueous Solutions

The stability of the cannabinoid prodrugs was studied in an aqueous sodium phosphate buffer solution (50 mM, ionic strength 0.5) at pH 6.8 and 37° C. The solutions of prodrugs were prepared by adding 10 μL of stock solution (10 mg/mL in DMSO) to the preheated buffer. The solutions were stirred at a constant temperature of 37° C. At appropriate intervals, 75 μL of samples were mixed with 75 μL of MeOH and analyzed for remaining prodrug by HPLC.

The pseudo-first-order rate constants (k_{obs}) and half-lives (t_{1/2}) for the degradation of the prodrugs were determined from the linear slopes of the plots by taking the logarithm of the remaining prodrug over time. Equations: k_{obs}=slope/−2.303 and t_{1/2}=ln2/k_{obs.}

The results are shown in Tables 3 and 5.

### Example 4: Stability in 80% Human Plasma (Serum)

The rates of the degradation of the prodrugs were determined in 80% human serum (pH 7.4) at 37° C. as an indication of the susceptibility of prodrugs to undergo bioconversion after reaching the circulation. The reactions were initiated by adding the first 10 μL of the prodrug stock solution (10 mg/mL in DMSO) to 200 μL preheated sodium phosphate buffer solution (50 mM, pH 7.4, ionic strength 0.5), and then adding 800 μL preheated human serum. The solutions were stirred at a constant temperature of 37° C. At appropriate intervals, 50 μL of samples were mixed with 150 μL of ice-cold MeOH. After immediate mixing and centrifugation (5 min at 14.000 rpm), the resulting clear supernatant was analyzed for the remaining prodrug by HPLC.

The results are shown in Tables 3 and 5.

### Example 5: Metabolic Stability

The prodrugs were incubated with the human liver microsomes at 37° C. as an indication of the susceptibility of prodrugs to undergo bioconversion in the liver. The reactions were initiated by adding 1.5 μL of the prodrug stock solution (10 mg/mL in DMSO) to a preheated solution consisting of 748.5 μL potassium phosphate buffer solution (20 mM, pH 7.4, ionic strength 0.5), 200 μL NADPH, and 50 μLhuman liver microsomes (mixed gender, 0.5 ml at 20 mg/mL). The solutions were stirred at a constant temperature of 37° C. At appropriate intervals, 50 μLof samples were mixed with 100 μL of ice-cold MeOH. After immediate mixing and centrifugation (5 min at 14.000 rpm), the resulting clear supernatant was analyzed for the remaining prodrug by HPLC.

The results are shown in Tables 3 and 5.

### Example 6: Aqueous Solubility and Apparent Partition Coefficient Measurements

CBD was dissolved in 50 mM phosphate buffer, pH 6.8, containing 10% (v/v) EtOH. Suspensions were centrifuged for 3 min at 14000 rpm. Solubilities are between 15-110 μg/ml. Some floating particles can be seen which, once dissolved, cause variation in the result. When the suspension was filtered (Millex 0.45 μm) no detectable (HPLC) CBD was found.

CBD solubility in various solvents when ca. 1 mg was dissolved in ca. 1 ml. Solutions/suspensions were centrifuged, and 100 μl of the sample was mixed with 100 μl MeOH:H₂O (1:1).

The solubility of CBD in various solvents is shown below:
1. 50% EtOH:50% H₂O=ca. 1 mg/ml
2. 15% HP-beta-CD:85% H₂O=0.7 mg/ml
3. 5% HP-beta-CD:0.5% HPMC:84.5% H₂O=0.16 mg/ml
4. 4% BSA:95% H₂O=0.07 mg/ml
5. 90% Propylene glycol:10% H₂O=0.87 mg/ml
6. 30% Solutol® HS 15:10% EtOH:60% H₂O=0.96 mg/ml

### Example 7: In Vitro Buccal Permeability Studies Method

Based on solubility studies using different solvents, 15% HP-beta-CD:85% H₂O was selected for the receiver compartment and 5% EtOH as a donor solvent in buccal permeation studies.

The in vitro permeability studies were carried out in commercial reconstructed human oral buccal epithelium cells (SkinEthic™ human oral epithelium). The SkinEthic™ HOE model is composed of TR146 cells (derived from a squamous cell carcinoma of the buccal mucosa) cultivated on an inert polycarbonate filter at the air-liquid interface in a chemically defined medium. This model forms an epithelial tissue devoid of stratum corneum, resembling histologically to the mucosa of the oral cavity.

Upon receiving the cells, each insert (0.5 cm² or 0.33 cm²) was placed in a plate containing 800 μL fresh medium at room temperature. The culture dishes were placed in the incubator at 37° C., 5% CO₂, and saturated humidity overnight. Next day, the inserts were placed in a plate containing 800 μL 15% HP-β-cyclodextrin in H₂O. The prodrugs were dissolved in 100% EtOH (5% (V/V) final conc.), and then sodium phosphate buffer solution (50 mM, pH 6.8, isotonic, 37° C.) was added (95% (V/V) final conc.). 400 μL of the prodrugs were applied as suspensions (ca. 2 mg/ml). The plates were stirred gently at 37° C. At appropriate intervals, the receptor compartment was sampled (100 μL) and replenished with fresh donor solution each time. The samples were mixed with 100 μL of MeOH, and analyzed for drug (both the parent cannabinoid and the prodrug) by HPLC-MS.

### Results

The buccal flux-values for the rate of delivery of drugs were determined from Fick's law of diffusion. Flux values of CBD, CBC, CBG, THC, and their prodrugs are shown in Tables 2 and 4.

Permeation of prodrugs (CBDp7, CBDp14, CBDp21, CBDp24, and CBDp27) in 5% ethanol suspension was better than that of CBD in 5% ethanol suspension (Table 2). Based on these results, the prodrugs CBDp24 (bis-3-pyrrolidine carboxylic acid ester prodrug) and CBDp27 (dimethylaminoethyl carbamate prodrug) were superior when compared to CBD and other prodrugs (Table 2). Another prodrug, CBDp14, bis-methylpiperazine carbamate prodrug showed also a significantly improved permeability compared to CBD. The calculated apparent partition coefficients and chemical and enzymatic stability of the prodrugs are shown in Table 3.

As shown in Table 4, the pyrrolidine ester prodrug of CBG (CBGp1) suspended in 5% ethanol showed 9.5 times improved permeability (flux value) compared to CBG suspended in 5% ethanol. Moreover, bis-methylpiperazine carbamate prodrug of CBG was 2.6 times better than CBG. The piperazine carbamate prodrug of THC (THCp3) was slightly better than THC.

### Example 8: In Vitro Skin Permeability Studies

### Method

Based on solubility studies using different solvents, 15% HP-beta-CD:85% H₂O was selected for receiver compartment and mineral oil or solvent mix as a donor solvent.

The in vitro permeability studies were carried out in commercial reconstructed human epidermis cells (SkinEthic™ Episkin Reconstructed human epidermis). This model is composed of TR146 cells (derived from a squamous cell carcinoma of the buccal mucosa) cultivated on an inert polycarbonate filter at the air-liquid interface in a chemically defined medium. This model forms an epithelial tissue devoid of stratum corneum, resembling histologically to the mucosa of the oral cavity.

Upon receiving the cells, each insert (0.5 cm² or 0.33 cm²) was placed in a plate containing 800 μL fresh medium at room temperature. The culture dishes were placed in the incubator at 37° C., 5% CO₂, and saturated humidity overnight. Next day, the inserts were placed in a plate containing 800 μL 15% HP-β-cyclodextrin in H₂O. CBD and CBG, and prodrugs CBDp27 and CBGp1 were dissolved in two different vehicles, 1:1:1 prop gly:water:EtOH/6% Brij 98, and 100% mineral oil. The receiver solution was 15% HP-beta-CD in phosphate buffer (pH 7.4). 400 μL the prodrugs were applied as suspensions (ca. 2 mg/ml). The plates were stirred gently at 37° C. At appropriate intervals, the receptor compartment was sampled (100 μL) and replenished with fresh donor solution each time. The samples were mixed with 100 μL of MeOH, and analyzed for drug (both the parent drug and prodrug) by HPLC-MS.

The flux-values for the rate of delivery of drugs were determined from Fick's law of diffusion.

### Results

The skin permeation (flux values) of CBD, CBG, and their prodrugs are shown in Table 6. When compared to the parent CBD molecule, CBDp27 is ˜140 fold more permeable in mineral oil and ˜40 fold more permeable in the solvent mix. The CBG prodrugs are ˜13 fold more permeable in mineral oil and >300 fold more permeable in the solvent mix. Overall, based on the permeability, both CBDp27 and pyrrolidine prodrug (CBGp1) has a higher potential to be used in pharmaceutical formulations.

TABLE 2
Buccal flux values of CBD and prodrugs

| Compound | Flux* (nmol/cm²/h) | Compared with CBD |
|---|---|---|
| CBD | 0.18 | |
| CBDp7 | 0.72 (1/3 CBD & 2/3 ) | 4-times better than CBD |
| | intact prodrug | |
| CBDp14 | 3.86 (only intact prodrug) | 21.4-times better than CBD |
| CBDp21 | 1.69 (only intact prodrug) | 9.4-times better than CBD |
| CBDp24 | 24.14 (mostly intact | 134.1-times better than CBD |
| | prodrug) | |
| CBDp27 | 21.24 (CBD + prodrug) | 118-times better than CBD |
| CBDp17 | 0.12 (only intact prodrug) | |
| CBDp18 | 0.07 (only intact prodrug) | |

*Flux is defined as nanomoles of compound moving through a 1 cm² area of buccal cell layer for one hour.

TABLE 3
log D_{6.8} values, hydrolysis and metabolic stability of CBD and CBD prodrugs

| | | | | | Kobs |
|---|---|---|---|---|---|
| | | Half-life in | | Half-life in | (based on |
| | | aqueous | Half-life in | human liver | microsomes |
| Compound | log D_{6.8} ^{a} | solution ^{b} | serum ^{c} | microsomes ^{d} | Half-life) |
| CBD | 6.32 | | | 0.50 (0.60, | 1.39 |
| | | | | 0.39) | |
| | | | | (avg. 0.50) | |
| | | | | CLint = 0.0231 ^{g} | |
| CBDp7 | 5.29 | 0.16 h | 2.6 h | 0.12 h | 5.77 |
| CBDp14 | 5.44 | 2.5 h ^{f} | stable | 0.86 h | 0.81 |
| | | | (no conversion | | |
| | | | in 6 h) | | |
| CBDp21 | 4.76 | not determined | not determined | not determined | N/A |
| CBDp24 | −0.2 | could not be | could not be | could not be | N/A |
| | | determined ^{e} | determined ^{e} | determined ^{e} | |
| CBDp27 | 4.44 | 0.14 h, 0.17 h | stable | 3.13; 3.13 | 0.22 |
| | | (pH 3 = stable, | (no conversion | (avg. 3.13) | |
| | | pH 4 = stable, | in 6 h) | | |
| | | pH 5 = stable, | | | |
| | | pH 7.4 = 0.87, | | | |
| | | pH 8 = 1.09, pH | | | |
| | | 9 = 1.64) | | | |

^{a} Partition coefficient at pH 6.8 (Log D_{6.8}) is calculated by MarvinSketch software
^{b} Chemical stability was determined in an aqueous buffer solution of pH 6.8
^{c} 80% human serum (pH 7.4) at 37° C.
^{d} Human liver microsomes as an indication of the susceptibility to undergo bioconversion in the liver.
^{e} Could not be determined due to fast degradation in standard and aqueous solutions,
^{f} Chemical stability can be significantly improved by replacing part of the aqueous solution with ethanol. 20% of ethanol in the solution resulted in a half-life of 50 hours.
^{g} Intrinsic clearance (CLint, ml/min/mg protein) was calculated based on CLint = k/P, where k is the elimination rate constant and P is the protein concentration in the incubation (1 mg).

TABLE 4
Buccal flux values of CBG, CBC, THC, and their prodrugs

| | | Flux compared |
|---|---|---|
| | | with parent |
| Compound | Flux* (nmol/cm²/h) | cannabinoid |
| CBG | 11.69 ± 0.11 | |
| CBGp1 | 111.79 ± 2.59 | 9.5-times better |
| | (CBG + prodrug) | than CBG |
| CBGp2 | 4.45 ± 0.45 (prodrug) | No improvement |
| | NO significant formation of CBG | |
| CBGp3 | 30.60 ± 2.02 (prodrug) | 2.6-times better |
| | NO significant formation of CBGp2 or | than CBG |
| | CBG | |
| CBGp5 | 10.31 ± 0.54 (prodrug) | No improvement |
| | NO significant formation of CBG | |
| CBC | 27.87 ± 2.46 | |
| CBCp1 | 15.88 ± 0.70 (prodrug) | No improvement |
| | NO significant formation of CBC | |
| CBCp3 | 5.74 ± 0.51 (prodrug) | No improvement |
| | NO significant formation of CBC | |
| THC | 19.65 ± 0.36 | |
| THCp1 | 19.22 ± 0.29 (prodrug) | No improvement |
| | NO significant formation of THC | |
| THCp3 | 31.30 ± 1.31 (prodrug) | 1.6-times better |
| | NO significant formation of THC | than THC |

*Flux is defined as nanomoles of compound moving through a 1 cm² area of buccal cell layer for one hour.

TABLE 5
log D_{6.8} values, hydrolysis and metabolic stability of CBG, CBC, THC, and their prodrugs

| | | | | | Kobs |
|---|---|---|---|---|---|
| | | Half-life in | | Half-life in | (based on |
| | | aqueous | Half-life in | human liver | microsomes |
| Compound | log D_{6.8} ^{a} | solution ^{b} | serum ^{c} | microsomes ^{d} | Half-life) |
| CBG | 7.04 | — | — | 1.61 (1.79, | 0.43 |
| | | | | 1.43) | |
| | | | | (avg. 1.61) | |
| | | | | CLᵢₙₜ = | |
| | | | | 0.00718 | |
| CBGp1 | 3.96 | 0.50; 0.54 | 0.70; 0.74 | 0.64; 0.67 | 1.05 |
| | | | | (avg. 0.66) | |
| CBGp2 | 6.60 | 1.14; 1.17 | No degradation | 1.62; 1.73 | 0.41 |
| | | | in 6 hrs | (avg. 1.68) | |
| CBGp3 | 6.16 | 0.28; 0.38 | No degradation | 0.73; 2.01; | 0.36 |
| | | | in 6 hrs | 2.95 | |
| | | | | (avg. 1.9) | |
| CBGp5 | 5.48 | 0.38; 0.26 | No degradation | 1.29; 1.79 | 0.45 |
| | | | in 6 hrs | (avg. 1.54) | |
| CBC | 6.60 | — | — | 1.86 (1.93, | 0.37 |
| | | | | 1.79) | |
| | | | | (avg. 1.86) | |
| | | | | CLᵢₙₜ = | |
| | | | | 0.00621 | |
| CBCp1 | 6.15 | 0.43; 0.20 | stable for 6 hrs | 4.18; 3.13 | 0.19 |
| | | | | (avg. 3.65) | |
| CBCp3 | 5.04 | 1.07; 0.22 | stable for 6 hrs | 7.16; 25.08; | 0.05 |
| | | | | 12.54 | |
| | | | | (avg. 14.93) | |
| THC | 5.95 | — | — | 0.18 (0.17, | 3.85 |
| | | | | 0.20) | |
| | | | | (avg. 0.18) | |
| | | | | CLᵢₙₜ = | |
| | | | | 0.06418 | |
| THCp1 | 5.50 | 0.66; 0.41 | No degradation | 2.64; 5.02 | 0.18 |
| | | | in 6 hrs | (avg. 3.83) | |
| THCp2 | 2.68 | 0.49; 0.46 | 0.42; 0.43 | 2.09; 2.79 | 0.28 |
| | | | | (avg. 2.44) | |
| THCp3 | 4.38 | 0.76; 1.07 | No degradation | 2.28; 5.02 | 0.19 |
| | | | in 5 hrs | (avg. 3.65) | |

^{a} Partition coefficient at pH 6.8 (Log D_{6.8}) is calculated by MarvinSketch software
^{b} Chemical stability was determined in an aqueous buffer solution of pH 6.8
^{c} 80% human serum (pH 7.4) at 37° C.
^{d} Human liver microsomes as an indication of the susceptibility to undergo bioconversion in the liver.

TABLE 6
In vitro skin permeability of CBD, CBG, and their prodrugs.

| | | | | Skin |
|---|---|---|---|---|
| | | | | permeation |
| No | Code | Structure | MW | (nmol/cm²/h) |
| CBD | | 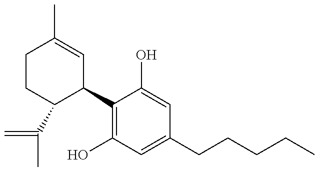 [SMILES: C=C(C)[C@@H]1CCC(C)=C[C@H]1c1c(O)cc(CCCCC)cc1O] | 314.47 | mineral oil: 0.0233 ± 0.007 solvent mix: 1.16 ± 0.12 |
| | CBDp27 | 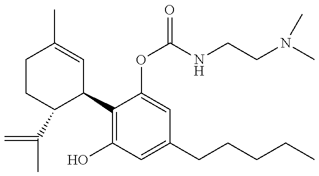 [SMILES: C=C(C)[C@@H]1CCC(C)=C[C@H]1c1c(O)cc(CCCCC)cc1OC(=O)NCCN(C)C] | 428.61 | mineral oil (prodrug): 3.28 ± 0.15 (≈140- fold better) solvent mix (prodrug): 47.42 ± 5.23 (≈40-fold better) mineral oil (CBD): no CBD solvent mix |
| | | | | (CBD): 0.10 ± |
| | | | | 0.01 |
| CBG | | 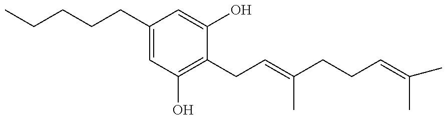 [SMILES: CCCCCc1cc(O)c(C/C=C(\C)CCC=C(C)C)c(O)c1] | 316.48 | mineral oil: 0.040 ± 0.029 solvent mix: 0.455 ± 0.127 |
| | CBGp1 | 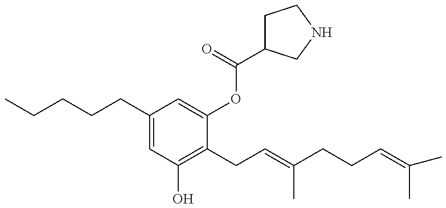 [SMILES: CCCCCc1cc(O)c(C/C=C(\C)CCC=C(C)C)c(OC(=O)C2CCNC2)c1] | 413.60 | mineral oil (prodrug): 0.54 ± 0.19 (≈13-fold better) solvent mix (prodrug): 148.0 ± 40.9 (>300- fold better) mineral oil (CBG): no CBG solvent mix (CBG): 0.46 ± 0.13 |

All citations are hereby incorporated by reference.

The present invention has been described with regard to one or more embodiments. However, it will be apparent to persons skilled in the art that a number of variations and modifications can be made without departing from the scope of the invention as defined in the claims. The scope of the claims should not be limited by the preferred embodiments set forth in the examples but should be given the broadest interpretation consistent with the description as a whole.

What is claimed is:

1. A cannabinoid prodrug having the formula: 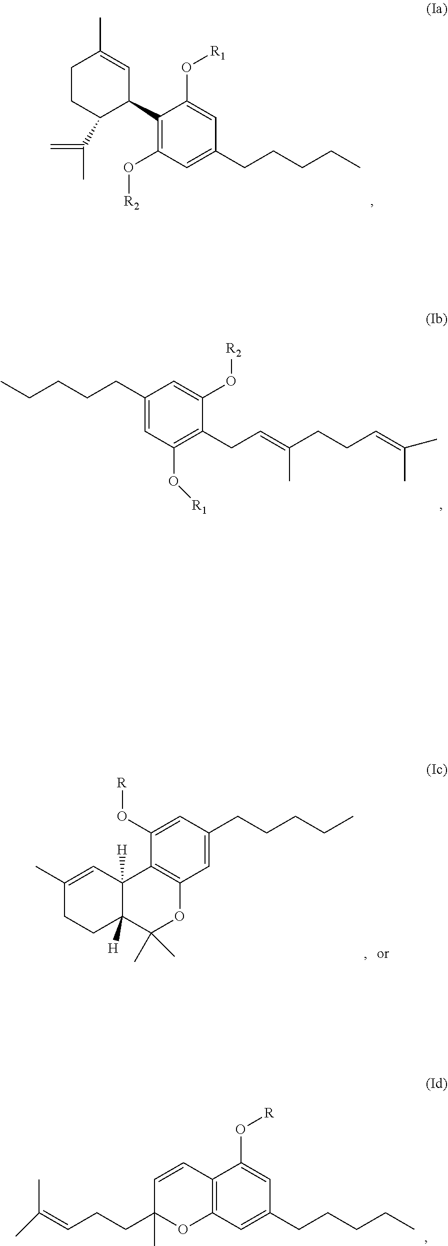
wherein:
R₁ and R₂ are each independently selected from hydrogen, alkyl, cycloalkyl, heterocycles, saturated heterocycles, or X, wherein X is selected from the group consisting of: 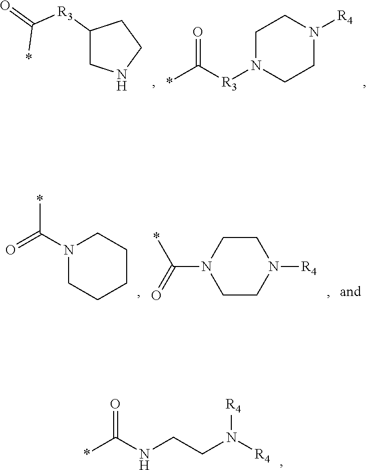
R₃ is a bond, C₁-C₈ alkyl, C₁-C₈ alkylene or C₁-C₈ alkylidene;
R₄ is hydrogen, or a C₁-C₆ alkyl.

2. The cannabinoid prodrug of claim 1, wherein R₃ is a bond or C₁-C₈ alkyl.

3. The cannabinoid prodrug of claim 1, wherein R₃ is a bond or methyl.

4. The cannabinoid prodrug of claim 1, wherein R₄ is hydrogen or C₁-C₆ alkyl.

5. The cannabinoid prodrug of claim 1, wherein R₄ is hydrogen or methyl.

6. The cannabinoid prodrug of claim 1, in the form of a free-base, salt, hydrate, enantiomer, isomer, tautomer, or derivative thereof.

7. The cannabinoid prodrug of claim 1 selected from the group consisting of:

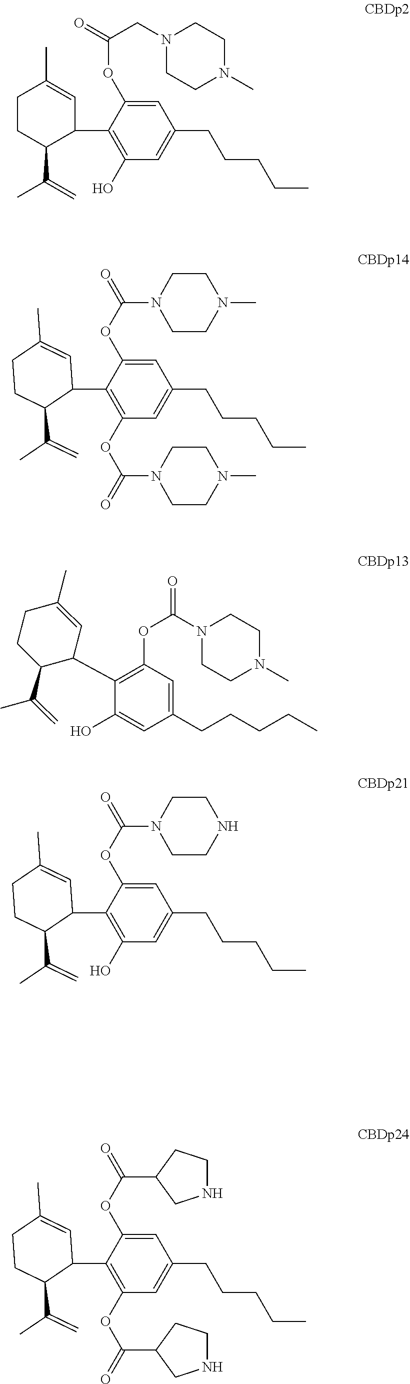

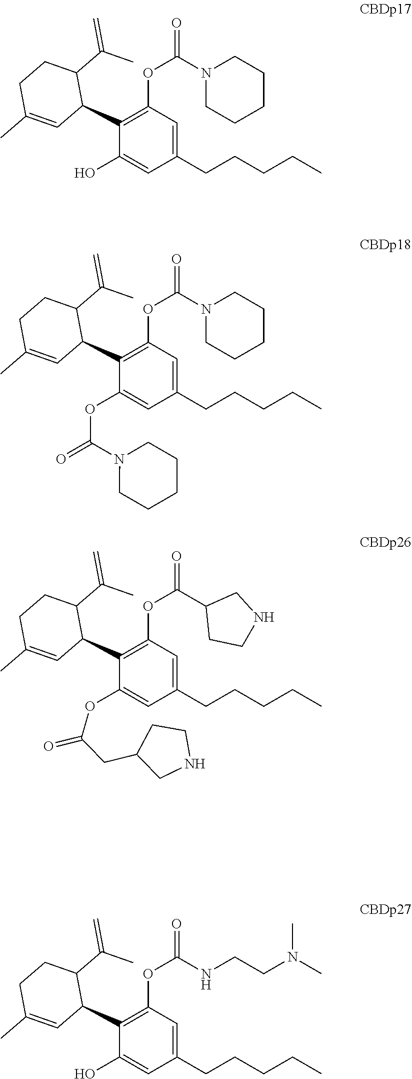

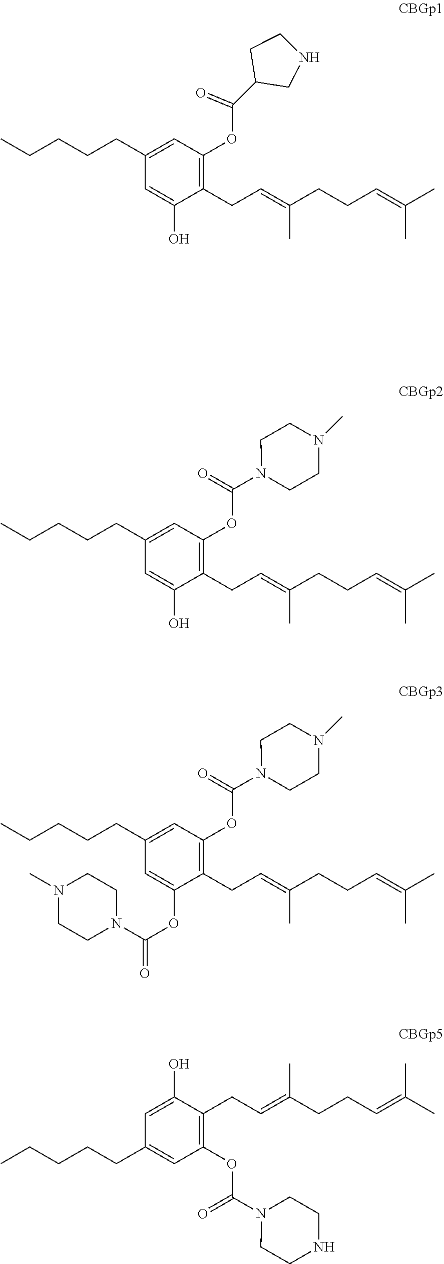

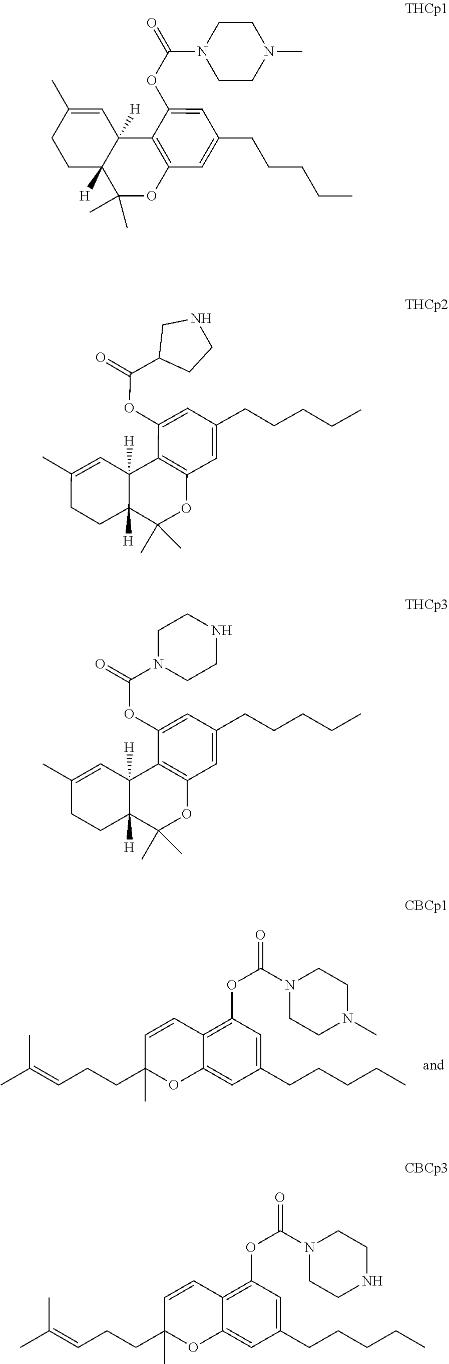

8. A composition comprising one or more cannabinoid prodrug according to claim 1, and one or more excipient or adjuvant.

9. The composition of claim 8, wherein the cannabinoid prodrug is present in the composition in an amount of between 0.1% and about 95% by weight.

10. The composition of claim 8, wherein the composition is a sustained, controlled or sustained and controlled release composition.

11. The composition according to claim 8, wherein the composition is as an analgesic, an anti-convulsant, an anti-psychotic, an anti-inflammatory, an anti-oxidant, a neuroprotective, an anti-cancer, or an immunomodulatory agent.

12. A method for treating a medical condition in a subject, the method comprising administering the cannabinoid prodrug as defined in claim 1 to the subject.

13. The method according to claim 12, wherein the medical condition is selected from the group consisting of: pain, epilepsy, an inflammatory condition, a psychotic disorder, a neurological disorder, an optical disorder, cancer, and an immunological disorder.

14. The method according to claim 12, wherein the cannabinoid prodrug is administered to the subject orally, transmucosally, transdermally, or parenterally.

15. The method according to claim 12, wherein the cannabinoid prodrug or composition is administered in a dose of from about 0.0001 mg/kg to about 2000 mg/kg.

16. The method according to claim 12, wherein the cannabinoid prodrug or composition is administered once a day or multiple times a day.

17. The method according to claim 12, wherein the cannabinoid prodrug or composition is administered between 0.0001-2000 mg/kg/day.

18. A method for the preparation of a cannabinoid prodrug according to claim 1, comprising: reacting a cannabinoid or cannabinoid derivative with a compound comprising the X moiety as defined in claim 1, to obtain the cannabinoid prodrug.

19. The method according to claim 18, wherein the cannabinoid is cannabidiol (CBD), tetrahydrocannabinol (THC), cannabichromene (CBC), or cannabigerol (CBG).

20. The method of claim 12, wherein the subject is a human or animal.
